(19) United States
(12) Patent Application Publication
(10) Pub. No.: US 2011/0189178 A1
(43) Pub. Date: Aug. 4, 2011

(54) Immunoprotection of Therapeutic Moieties Using Enhanced Fc Regions

(75) Inventors: John Desjarlais, Pasadena, CA (US); Gregory L. Moore, Monrovia, CA (US); Holly M. Horton, Pasadena, CA (US)

(73) Assignee: XENCOR, INC., Monrovia, CA (US)

(21) Appl. No.: 13/021,638

(22) Filed: Feb. 4, 2011

Related U.S. Application Data
(60) Provisional application No. 61/301,511, filed on Feb. 4, 2010.

(51) Int. Cl. A61K 39/395; C07K 19/00; C07H 21/00; C12N 5/10; C12P 21/00; A61P 37/06

(52) U.S. Cl. 424/133.1; 424/178.1; 530/391.7; 536/23.4; 435/325; 435/69.3; 435/69.6

(57) ABSTRACT

The present application relates to therapeutic moieties displaying reduced immunogen response, particularly for therapeutic purposes.

This application claims benefit under 35 U.S.C. §119(e) to U.S. Ser. No. 61/301,511, filed Feb. 4, 2010; entirely incorporated herein by reference.

### FIELD OF THE INVENTION

The present application relates to therapeutic moieties displaying reduced immunogen response, particularly for therapeutic purposes.

### BACKGROUND OF THE INVENTION

The humoral immune response requires antigen-specific B cell activation and subsequent terminal differentiation into plasma cells. Engagement of B cell antigen receptor (BCR) on mature B cells activates an intracellular signaling cascade, including calcium mobilization, that leads to cell proliferation and differentiation. Coengagement by immune complex of BCR with the inhibitory Fc receptor FcγRIIb, the only IgG receptor expressed on B cells, inhibits B cell activation signals through a negative feedback loop.

Antigen recognition by B cells is mediated by the B cell receptor (BCR), a surface-bound immunoglobulin in complex with signaling components CD79a (Igα) and CD79b (Igβ). Crosslinking of BCR upon engagement of immune-complexed antigen results in phosphorylation of immunoreceptor tyrosine-based activation motifs (ITAMs) within CD79a and CD79b, initiating a cascade of intracellular signaling events that recruit downstream molecules to the membrane and stimulate calcium mobilization. This leads to the induction of diverse B cell responses including cell survival, proliferation, and differentiation (1-3). Other components of the BCR coreceptor complex enhance (e.g., CD19, CD21, and CD81) or suppress (e.g., CD22 and CD72) BCR activation signals (4, 5). In this way the immune system maintains multiple BCR regulatory mechanisms to ensure that B cell responses, including antibody production and antigen presentation, are tightly controlled.

When antibodies are produced to an antigen, the circulating level of specific immune complexes increases. These immune complexes neutralize antigen-induced B cell activation by coengaging cognate BCR with the low-affinity inhibitory receptor FcγRIIb, the only IgG receptor on B cells (6). This negative feedback of antibody production requires interaction of the antibody Fc domain with FcγRIIb, because immune complexes containing F(ab′)2 antibody fragments are not active (7). The intracellular immunoreceptor tyrosine-based inhibitory motif (ITIM) of FcγRIIb is necessary to inhibit BCR-induced intracellular signals (8, 9). This inhibitory effect occurs through phosphorylation of the FcγRIIb ITIM, which recruits Src homology region 2-containing inositol polyphosphate 5-phosphatase (SHIP) to neutralize ITAM-induced intracellular calcium mobilization (3, 10, 11). In addition, FcγRIIb-mediated SHIP phosphorylation inhibits the downstream Ras-MAPK proliferation pathway (12).

The importance of FcγRIIb in negative regulation of B cell responses has been demonstrated using FcγRIIb-deficient mice, which fail to regulate humoral responses (13), are sensitized to collagen-induced arthritis (14), and develop lupus-like disease (15, 16) and Goodpasture's syndrome (17). FcγRIIb dysregulation has also been associated with human autoimmune disease; for example, alleles of polymorphisms in the promoter (18, 19) and transmembrane domain (20-22) of FcγRIIb have been linked with increased prevalence of systemic lupus erythematosus (SLE). SLE patients also show reduced FcγRIIb surface expression on B cells (23, 24) and, as a consequence, exhibit dysregulated signaling (23). The pivotal role of FcγRIIb in regulating B cells, supported by mouse models and clinical evidence, makes it an attractive target for controlling immune response and treating autoimmune and inflammatory disorders (3, 25, 26).

### SUMMARY OF THE INVENTION

According, the present invention provides methods of reducing a B-cell mediated immune response to a protein comprising administering to a patient in need thereof a fusion composition comprising a first domain comprising the protein and a second domain comprising an Fc variant of a human wild-type Fc region that binds the FcγRIIb receptor with a Kd of less than about 100 nM. The protein can be a therapeutic protein, an autoantigen, and/or an allergen.

In an additional aspect, the invention provides methods of reducing a B-cell mediated immune response to a therapeutic antibody comprising administering to a patient in need thereof a variant therapeutic antibody, wherein the Fc domain of said therapeutic antibody comprises an Fc variant of a human wild-type Fc region that binds the FcγRIIb receptor Kd of less than about 100 nM.

In an additional aspect, the invention provides fusion compositions comprising a first fusion protein comprising a protein selected from the group consisting of an autoantigen, an allergen and a non-antibody therapeutic protein and a second fusion protein comprising an Fc variant of a human wild-type Fc region that binds the FcγRIIb receptor with a Kd of less than about 100 nM.

In a further aspect, the Fc variants above comprises an amino acid substitution selected from the group consisting of 234D, 234E, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E, wherein numbering is according to the EU index.

In an additional aspect, the Fc variant comprises an amino acid substitution selected from the group consisting of 235Y, 236D, 239D, 266M, 267E, 268D, 268E, 328F, 328W, and 328Y wherein numbering is according to the EU index.

In a further aspect, the Fc variant comprises an amino acid substitutions selected from the group consisting of 235Y/267E, 236D/267E, 239D/268D, 239D/267E, 267E/268D, 267E/268E, and 267E/328F, wherein numbering is according to the EU index.

In an additional aspect, the invention provides methods of treating a B-cell mediated autoimmune disease comprising administering to a patient in need thereof a fusion composition comprising an autoantigen first protein associated with said autoimmune disease and a second protein comprising an Fc variant of a human wild-type Fc region that binds the FcγRIIb receptor Kd of less than about 100 nM.

In a further aspect, in a method of treatment comprising the administration of a therapeutic antibody to a patient in need thereof, the improvement comprising administering a variant therapeutic antibody wherein the Fc region of said antibody is an Fc variant of a human wild-type Fc region that binds the FcγRIIb receptor with a Kd of less than about 100 nM.

In an additional aspect, in a method of treatment comprising the administration of a therapeutic protein to a patient in need thereof, the improvement comprising administering a fusion therapeutic protein comprising the therapeutic protein and an Fc variant of a human wild-type Fc region that binds the FcγRIIb receptor with a Kd of less than about 100 nM.

### BRIEF DESCRIPTION OF THE DRAWINGS

FIG. 1. Immunoprotection mechanism for therapeutic proteins. A critical step in immune response to an administered therapeutic protein (TP) is engagement of the B cell receptor (BCR) and subsequent activation of B cells. Fusion of the therapeutic protein to an Fc region enhanced in affinity (with high affinity) for FcγRIIb (IIbE), but not to a native (weak affinity) Fc region, is able inhibit B cell activation and immune response.

FIG. 2. Adalimumab elicits a strong immune response in cynomolgus monkeys. The data show the time course of anti-drug antibody (ADA) response in two monkeys following single dose 4 mg/kg intravenous administration of adalimumab IgG1. The cutpoint of the ADA assay was approximately 200 RFU (relative fluorescence units). The RFU's for the two monkeys on the final day of the study (day 59) were 38416 and 66518 respectively. The mean of 52467 RFU is 262-fold relative to cutpoint, representing quantitatively the immune response to the administered biotherapeutic.

FIG. 3. Adalimumab elicits a strong immune response in mice. The data show the time course of anti-drug antibody (ADA) response in four C57BL/6 mice following single dose 2 mg/kg intravenous administration of adalimumab IgG1. The top panel shows the ADA response for individual mice and the bottom panel shows the group mean average and standard error. The cutpoint of the ADA assay was approximately 200 RFU (relative fluorescence units). The mean RFU for the four mice on the final day of the study (day 14) was 12302. This level is 62-fold relative to cutpoint, representing quantitatively the immune response.

FIG. 4. [SEQ ID NOS: 1-4] FIG. 4 provides amino acid sequences of the light and heavy chanins of IgG1 and Fc-engineered adalimumab.

FIG. 5. Sensorgrams for binding to FcγRIIb.

FIG. 6. Immunoprotective Fc region (IIbE) inhibits ADA response to adalimumab relative to a native IgG1 Fc region in mice transgenic for human FcγRIIb (hCD32b+tg mice). The data show the time course of anti-drug antibody (ADA) response in five transgenic mice following single dose 2 mg/kg intravenous administration of adalimumab IgG1 and variant (IIbE). The top panel shows the ADA response for individual mice and the bottom panel shows the group mean average and standard error. Mean ADA response on the final day of the study (day 14) was 56640 for IgG1 and 5877 for IIbE. The cutpoint of the ADA assay was approximately 200 RFU (relative fluorescence units). The mean RFU's for the five mice for the IgG1 and IIbE groups on the final day of the study (day 14) were thus 283-fold and 29-fold relative to cutpoint respectively. Thus reduction in ADA between IgG1 (RFU=56640) and IIbE (RFU=5877) was 90%, and thus the IIbE Fc region resulted in a 10-fold reduction in ADA response.

FIG. 7. Immunoprotective Fc region (IIbE) inhibits ADA response to adalimumab relative to a native IgG1 Fc region in mice transgenic for human FcγRIIb (hCD32b+tg mice) but not in genetically matched mice lacking human FcγRIIb (hCD32b−tg mice). The data show the time course of anti-drug antibody (ADA) response in transgenic mice either containing (hCD32b+tg) or lacking (hCD32b−tg) human FcγRIIb. Mice were adminstered a single 10 mg/kg dose of antibody intravenously. The top panel shows the ADA response for individual mice and the bottom panel shows the group mean average and standard error. Mean ADA response on the final day of the study (day 24) for the hCD32b+tg mice was 218478 for IgG1 and 92092 for IIbE. The cutpoint of the ADA assay was approximately 200 RFU (relative fluorescence units). The mean RFU's for the five mice for the IgG1 and IIbE groups on the final day were thus 1092-fold and 460-fold relative to cutpoint respectively. Thus reduction in ADA between IgG1 (RFU=218478) and IIbE (RFU=92092) was 58%, and thus the IIbE Fc region resulted in a 2.4-fold reduction in ADA response.

FIG. 8. Amino acid sequences of the Fc regions of the native human IgG isotypes and the Fc-engineered 267E/328F IIbE IgG1 version described in the Examples [SEQ ID NOS: 5-8].

FIG. 9. Immunoprotection mechanism for application to reducing the immune response to autoantigens and allergens. An important step in the pathology of many autoimmune and allergic disease is activation of B cells by autoantigen (A) or allergen (A) via engagement of the B cell receptor (BCR). Fusion of the autoantigen or allergen to an Fc region enhanced in affinity (with high affinity) for FcγRIIb (IIbE), but not to a native (weak affinity) Fc region, is able to inhibit B cell activation and immune response.

FIG. 10. Amino acid sequences of MOG and MOG peptide Fc fusions with native, FcγRIIb-enhanced (IIbE), and FcγR-knockout (KO) Fc regions [SEQ ID NOS: 9-14].

FIG. 11. Binding of human MOG-Fc fusions to human FcγRIIb, human V158 FcγRIIIa, and anti-MOG antibody as measured by Biacore. Fc regions of the MOG-Fc fusions contained either native IgG1, an FcγRIIb-enhanced (IIbE) variant 267E/328F, or a knockout (KO) variant 236R/328R.

FIG. 12. Affinities of Fc variant antibodies for human FcγRs as determined by Biacore surface plasmon resonance. The table lists the dissociation constant (Kd) for binding anti-CD19 variant antibodies to human FcγRI, FcγRIIa (131R), FcγRIIa (131H), FcγRIIb, FcγRIIIa (158V), and FcγRIIIa (158F). Multiple observations have been averaged. n.d.=no detectable binding.

FIG. 13. Fold affinities of Fc variant antibodies for human FcγRs as determined by Biacore surface plasmon resonance. The table lists the fold improvement or reduction in affinity relative to WT IgG1 for binding of anti-CD19 variant antibodies to human FcγRI, FcγRIIa (131R), FcγRIIa (131H), FcγRIIb, FcγRIIIa (158V), and FcγRIIIa (158F). Fold=KD (Native IgG1)/KD (variant). n.d.=no detectable binding.

### DETAILED DESCRIPTION OF THE INVENTION

### Overview

The humoral immune response requires antigen-specific B cell activation and subsequent terminal differentiation into plasma cells. Engagement of B cell antigen receptor (BCR) on mature B cells activates an intracellular signaling cascade, including calcium mobilization, which leads to cell proliferation and differentiation. This leads to the production of antibodies against immunogens that engage the BCR complex. Thus, B-cell receptor (BCR) induced B cell proliferation can lead to unwanted immune responses, particularly humoral immune responses. For example, the administration of a number of therapeutic drugs, including proteinaceous therapeutic drugs as are more fully described below, can lead to immune responses to the drug itself, leading to both a loss of drug efficacy as well as the potential for significant side effects. Similarly, this same immune response can be seen to autoantigens in autoimmune diseases.

However, coengagement by immune complex of BCR with the inhibitory Fc receptor FcγRIIb, the only IgG receptor expressed on B cells, inhibits B cell activation signals through a negative feedback loop. FcγRIIb-mediated inhibition of BCR-stimulated B cell activation occurs upon engagement of the FcγRIIb receptor at high affinity, which is result of avidity of IgG/antigen immune complexes. Under physiological conditions, bridging of the BCR with FcγRIIb and subsequent B cell suppression occurs via immune complexes of IgGs and cognate antigen.

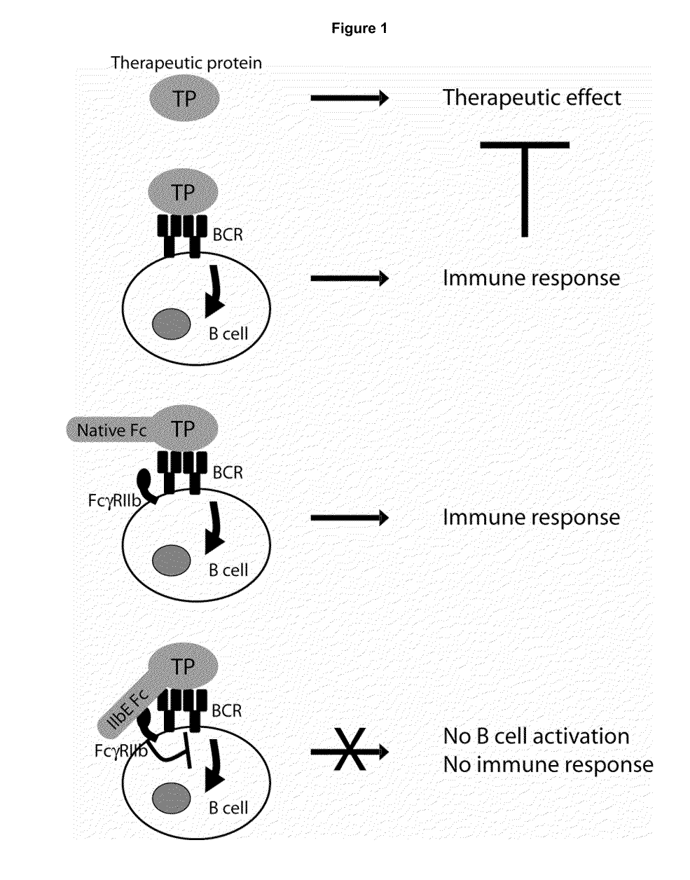

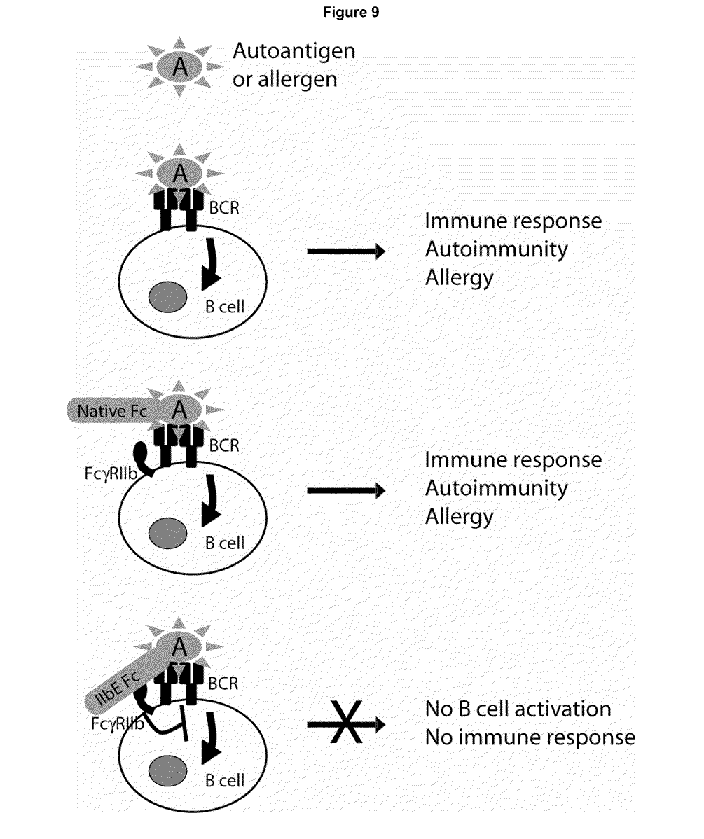

The present invention is drawn to the use of the FcγRIIb-mediated inhibitory mechanism to reduce the immune response to a therapeutic protein, autoantigens, and/or allergen. Our novel approach, illustrated in FIGS. 1 and 9, mimics the inhibitory effects of immune complex by high-affinity coengagement of FcγRIIb and the BCR coreceptor complex on human B cells. A key step in immune response to a protein is engagement with anti-immunogen specific BCR on B cells followed by activation, internalization, and presentation to T cells. Fusion of the protein to the IgG Fc region enables interaction with the inhibitory receptor FcγRIIb. However, because native IgG's bind FcγRIIb with weak (uM) affinity, FcγRIIb-mediated inhibition occurs in response only to immune complexed but not monomeric IgG Fc.

Central to the invention is the generation of a high affinity interaction between the Fc region and FcγRIIb, which may enable maximal inhibition of B cell activation by monovalent (non-immune complexed) therapeutic protein. Coupling of FcγRIIb-enhanced (IIbE) Fc domains to immunogens such as therapeutic proteins, autoantigens, and/or allergens utilizes the natural inhibitory pathway to inhibit the B cell response to the fusion partner. Enhanced affinity of the protein-Fc fusion for the inhibitory FcγRIIb prevents anti-therapeutic protein, anti-autoantigen, and/or anti-allergen B cells from activation and differentiation into immunoglobulin-producing plasma cells.

Thus, the present invention is directed in some aspects to the coupling of an immunogen to a variant Fc region that has been engineered to bind with increased binding affinity to the FcγRIIb receptor to result in significant “immunoprotection”, such that the administration of the previously immunogenic moiety, e.g. the immunogen, reduces or prevents the production of antibodies. Essentially, the addition of the variant Fc domain forces a “tolerization” of the immunogen to which it is attached, thus silencing the response. The immunogen to which the variant Fc domain is attached can be an exogenous immunogen, such as a therapeutic drug, including therapeutic proteins and antibodies or allergens, or an endogenous immunogen such as an autoantigen, as are further described below.

We have engineered the Fc domain to create a single biologic with high affinity for FcγRIIb that mimics the suppressive effects of cognate immune complex on activated B cells (U.S. Ser. No. 12/156,183, filed May 30, 2008, entitled “Methods and Compositions for Inhibiting CD32b Expressing cells”, herein incorporated expressly by reference). B cells are important in adaptive immunity because BCR-antigen complex internalization is the first step in antigen presentation. Because FcγRIIb coengagement inhibits BCR-dependent antigen internalization and processing, the activities of the IIbE variants presented here may also suppress T cell-mediated adaptive immunity. Thus, an application of the IIbE Fc domain is as a fusion with an immunogenic therapeutic protein. Such a fusion will suppress differentiation, survival, and proliferation only of B cell populations possessing BCRs specific for epitopes of the immunogen. This strategy thus selectively eliminates only drug-reactive B cells, and thus mitigates potential immunogenicity concerns arising from the clinical use of therapeutic proteins. Thus IIbE Fc regions have therapeutic applications by selectively eliminating only immunogen-reactive B cells.

Coupling of FcγRIIb-enhanced Fc domains to therapeutic proteins has several clinical benefits. First, the enhanced affinity of the protein-Fc fusion for the inhibitory FcγRIIb will prevent anti-therapeutic B cells from activation and differentiation into immunoglobulin-producing plasma cells. Second, the Fc domain also generally serves to improve the pharmacokinetic properties of the therapeutic protein, a strategy that has been applied successfully with a variety of agents.

Fusion of these variant Fc domains to therapeutic proteins has widespread utility in the treatment of human diseases. Immunogenicity is a foremost issue in the development of biologics therapies, sometimes resulting in the simple but commercially damaging loss of efficacy, as with many of the murine-derived antibodies that have failed clinically due to high frequency of human anti-mouse antibody (HAMA) responses. Occasionally, an anti-therapeutic immune response results in serious long-term medical problems, such as the pure red cell aplasia attributed to immune responses against endogenous erythropoietin, caused by treatment with recombinant erythropoietins such as Epogen, Procrit, and Eprex.

In addition, this same mechanism finds use for other immunogens in addition to therapeutic proteins (including antibodies). In these embodiments, fusions of the variant Fc domain with either autoantigens or allergens can be made. Both of these types of molecules can cause unwanted immune effects. Thus, by coupling the B-cell suppressive effect of the variant Fc domain with an immunogen that causes unwanted effects, e.g. autoimmune disease or inflammation/allergic responses, the response to the immunogen is reduced.

When the therapeutic drug is a therapeutic antibody, the endogenous Fc domain of the therapeutic antibody is altered to contain the amino acid substitutions that result in increased FcγRIIb, as is described below. Alteration of one or more specific amino acids in the Fc domain of the Fc region to significantly increase the binding of the variant Fc domain to the FcγRIIb receptor, can result in the reduction and/or elimination of humoral antibodies to the therapeutic antibody.

We chose as our test system for the immunoprotection approach the anti-TNFα (anti-TNF) antibody adalimumab (marketed as the drug Humira®). Despite being engineered as a fully human amino acid sequence, Humira is known to elicit significant immunogenicity in humans. Approximately 5.5% of patients with rheumatoid arthritis under combination treatment with adalimumab and methotrexate (MTX) developed an unwanted immune response, and without concomitant methotrexate the incidence of an unwanted immune response was 12.4% (European Medicines Agency, 2008, European Public Assessment Report (EPAR) for Humira®). Immunogenicity of adalimumab negatively affects its clinical outcome (West et al., 2008, Alimentary Pharmacology & Therapeutics 28:1122-1126.

When the therapeutic drug is not an antibody but a therapeutic protein, the therapeutic protein is fused to the variant Fc domain. This fusion can occur in a number of ways, including but not limited to a genetic linkage, a chemical conjugation or the fusion of the N-terminus of one component (e.g. the variant Fc domain) to the C-terminus of the other component (e.g. the therapeutic protein), or vice versa, either directly or through the use of traditional linkers as is described below.

The immunogen that is fused to an Immunoprotective Fc domains may be an autoantigen or allergen that plays a role in a disease. In this case the adminstered biotherapeutic may mimic the suppressive effects of cognate immune complex on activated B cells. Because FcγRIIb coengagement inhibits BCR-dependent antigen internalization and processing, the activities of the IIbE variants presented here may also suppress T cell-mediated adaptive immunity, thereby inhibiting the underlying biology of the disease.

Whereas particular embodiments of the invention have been described above for purposes of illustration, it will be appreciated by those skilled in the art that numerous variations of the details may be made without departing from the invention as described in the appended claims. All references cited herein are incorporated in their entirety.

Accordingly, the present invention provides methods of preventing a B-cell mediated immune response to a number of different types of proteins.

B-Cell Mediated Immune Responses

By “a B-cell mediated immune response” herein is meant the antigen-specific B cell activation by engagement of the B cell antigen receptor on mature B cells which results in an intracellular signaling cascade, including calcium mobilization, that leads to cell proliferation and differentiation. In general, there are a variety of assays to assess the presence or absence of a B-cell mediated immune response. As is described in the examples, one assay is the anti-drug antibody (ADA) assay which can be run in either mice or non-human primates such as cynomolgus monkeys. In general, for immunogenicity of a biotherapeutic, a B-cell mediated immune response is determined to occur when the level of pre-clinical (for example mouse or monkey) or clinical (human) anti-drug antibody is greater than the cutpoint of an ADA assay. An ADA response may for example be 2-fold above cutpoint, although preferably it is greater than 10-fold above cutpoint. As will also be appreciated by those in the art, when the present invention is used on an already approved therapeutic drug, the reduction of the immune response may be seen in human patients. In general, immune response to an antigen or allergen is determined as the observation of disease in a pre-clinical model of the disease, for example in mice or monkeys, when the animals are administered the antigen or allergen. In this case immune response is typically measured using some disease score that may reflect incidence, severity, or other metrics, and will vary depending on the disease and pre-clinical model as is established in the art.

The invention provides for the prevention of B-cell mediated immune responses. By “prevention” herein is meant a reduction in the B-cell mediated immune response. As will be appreciated by those in the art, the invention finds use even if a complete elimination of the B-cell mediated immune response in the test animals and/or patients does not occur. Reduction in immune response may be a reduction in ADA response where the application is to reduce immunogenicity of a biotherapeutic, or may be a reduction in disease score in a preclinical model of the disease or in humans who suffer from the disease. Included within the definition of “prevention” or “reduction” of the B-cell mediated immune response is a reduction in by at least 2-fold, with reduction of at least about 2- to 10-fold being useful and reductions of at least about 10-fold being similarly useful.

However, it should be appreciated that the present invention finds use in situations where a therapeutic protein (including a therapeutic antibody) has not yet been shown to cause a significant immune response. That is, the incorporation of the amino acid substitutions with the Fc domain can be done prior to an immune response being a problem with an administered drug. In this case, the reduction of a B-cell mediated immune response is determined by comparing the response in an animal model, such as the ADA models outlined above.

The present invention provides methods of reducing the immune response of immunogens.

Immunogens

As is described herein, the present invention provides methods for reducing the B-cell mediated immune response to an immunogen. In general, an “immunogen” is a substance that causes an immune response, in this case an undesirable B-cell mediated immune response, in a patient. That is, the immunogen can be a protein involved in mediating the pathology of a disease. As is outlined herein, in general, immunogens fall into four general categories herein. In some embodiments, described herein, the immunogen is a therapeutic antibody (e.g. the immunogen is exogenous to the host). In some embodiments, the immunogen is a therapeutic protein (again an exogeneous immunogen). In other embodiments, the immunogen is an autoantigen, resulting in an immune response to an endogenous molecule, leading to an undesirable autoimmune disease or symptoms. In still further embodiments, the immunogen is an allergen (again an exogenous molecule to the host), which normally causes an undesirable allergic reaction in some patients.

Antibodies

In some embodiments, the immunogen is a therapeutic antibody. That is, as described below and known in the art, therapeutic antibodies can take on a number of formats. In the context of this invention, a therapeutic antibody has an antigen-binding domain as well as an Fc domain. Therapeutic antibody structures that no longer possess Fc domains can also be used (e.g. Fabs), but in that case they would be considered “therapeutic proteins” to which a variant Fc domain of the invention would be fused, creating a fusion composition herein.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Thus, “isotype” as used herein is meant any of the subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE. It should be understood that therapeutic antibodies can also comprise hybrids of isotypes and/or subclasses. For example, hybrid IgG1/IgG2 hybrids are described in U.S. Publication No. 2006/0134150, herein incorporated by reference in its entirety.

The amino-terminal portion of each chain includes a variable region of about 100 to 110 or more amino acids primarily responsible for antigen recognition. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230.

Of particular interest in the present invention are the Fc regions. By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. Fc may refer to this region in isolation, or this region in the context of an Fc fusion (“fusion composition” or “fusion construct”), as described below. Fc domains include all or part of an Fc region; that is, N- or C-terminal sequences may be removed from wild-type or variant Fc domains recited herein, as long as binding to FcγRIIb is preserved. That is, an Fc domain of the invention retains binding to FcγRIIb, with variant Fc domains having binding affinities and/or increased binding to the FcγRIIb receptor as outlined herein. Fc polypeptides include antibodies, Fc fusions, isolated Fcs, and Fc fragments.

In some embodiments, the antibodies are full length. By “full length antibody” herein is meant the structure that constitutes the natural biological form of an antibody, including variable and constant regions, including one or more modifications as outlined herein.

Alternatively, the antibodies can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively. Again, to the extent the antibody no longer possesses an Fc domain it would fall under the definition of a therapeutic protein for fusion to the variant Fc regions of the invention.

In one embodiment, the antibody is an antibody fragment. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546, entirely incorporated by reference) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (viii) bispecific single chain Fv (WO 03/11161, hereby incorporated by reference) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference).

Chimeric and Humanized Antibodies

In some embodiments, the antibody can be a mixture from different species, e.g. a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213, all entirely incorporated by reference). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res.57(20):4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference. In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

Again, it should be understood that antibodies that are “humanized” or “human” can still elicit significant immune responses and thus the present invention can be used to mask that response.

In one embodiment, the antibodies of the invention multispecific antibody, and notably a bispecific antibody, also sometimes referred to as “diabodies”. These are antibodies that bind to two (or more) different antigens. Diabodies can be manufactured in a variety of ways known in the art (Holliger and Winter, 1993, Current Opinion Biotechnol. 4:446-449, entirely incorporated by reference), e.g., prepared chemically or from hybrid hybridomas.

In one embodiment, the antibody is a minibody. Minibodies are minimized antibody-like proteins comprising a scFv joined to a CH3 domain. Hu et al., 1996, Cancer Res. 56:3055-3061, entirely incorporated by reference. In some cases, the scFv can be joined to the Fc region, and may include some or the entire hinge region.

Antibody Modifications

In addition to the modification of the Fc region of an antibody, other modifications can be made. For example, the molecules may be stabilized by the incorporation of disulphide bridges linking the VH and VL domains (Reiter et al., 1996, Nature Biotech. 14:1239-1245, entirely incorporated by reference). In addition, there are a variety of covalent modifications of antibodies that can be made as outlined below.

Covalent modifications of antibodies are included within the scope of this invention, and are generally, but not always, done post-translationally. For example, several types of covalent modifications of the antibody are introduced into the molecule by reacting specific amino acid residues of the antibody with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues may also be derivatized by reaction with bromotrifluoroacetone, α-bromo-3-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole and the like.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidazole and tetranitromethane are used to form O-acetyl tyrosyl species and 3-nitro derivatives, respectively. Tyrosyl residues are iodinated using 125I or 131I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking antibodies to a water-insoluble support matrix or surface for use in a variety of methods, in addition to methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440, all entirely incorporated by reference, are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983], entirely incorporated by reference), acetylation of the N-terminal amine, and amidation of any C-terminal carboxyl group.

In addition, as will be appreciated by those in the art, labels (including fluorescent, enzymatic, magnetic, radioactive, etc. can all be added to the antibodies (as well as the other compositions of the invention).

Glycosylation

Another type of covalent modification is glycosylation. In another embodiment, the antibodies comprising variant Fc domains disclosed herein can be modified to include one or more engineered glycoforms. By “engineered glycoform” as used herein is meant a carbohydrate composition that is covalently attached to the antibody, wherein said carbohydrate composition differs chemically from that of a parent antibody. Engineered glycoforms may be useful for a variety of purposes, including but not limited to enhancing or reducing effector function. Engineered glycoforms may be generated by a variety of methods known in the art (Umaria et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473; U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1, all entirely incorporated by reference; (Potelligent® technology [Biowa, Inc., Princeton, N.J.]; GlycoMAb® glycosylation engineering technology [Glycart Biotechnology AG, Zurich, Switzerland]). Many of these techniques are based on controlling the level of fucosylated and/or bisecting oligosaccharides that are covalently attached to the Fc region, for example by expressing an IgG in various organisms or cell lines, engineered or otherwise (for example Lec-13 CHO cells or rat hybridoma YB2/0 cells), by regulating enzymes involved in the glycosylation pathway (for example FUT8 [α1,6-fucosyltranserase] and/or β1-4-N-acetylglucosaminyltransferase III [GnTIII]), or by modifying carbohydrate(s) after the IgG has been expressed. Engineered glycoform typically refers to the different carbohydrate or oligosaccharide; thus an IgG variant, for example an antibody or Fc fusion, can include an engineered glycoform. Alternatively, engineered glycoform may refer to the IgG variant that comprises the different carbohydrate or oligosaccharide. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antibody amino acid sequence is preferably altered through changes at the DNA level, particularly by mutating the DNA encoding the target polypeptide at preselected bases such that codons are generated that will translate into the desired amino acids.

Another means of increasing the number of carbohydrate moieties on the antibody is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 and in Aplin and Wriston, 1981, CRC Crit. Rev. Biochem., pp. 259-306, both entirely incorporated by reference.

Removal of carbohydrate moieties present on the starting antibody may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131, both entirely incorporated by reference. Enzymatic cleavage of carbohydrate moieties on polypeptides can be achieved by the use of a variety of endo- and exo-glycosidases as described by Thotakura et al., 1987, Meth. Enzymol. 138:350, entirely incorporated by reference. Glycosylation at potential glycosylation sites may be prevented by the use of the compound tunicamycin as described by Duskin et al., 1982, J. Biol. Chem. 257:3105, entirely incorporated by reference. Tunicamycin blocks the formation of protein-N-glycoside linkages.

Another type of covalent modification of the antibody comprises linking the antibody to various nonproteinaceous polymers, including, but not limited to, various polyols such as polyethylene glycol, polypropylene glycol or polyoxyalkylenes, in the manner set forth in, for example, 2005-2006 PEG Catalog from Nektar Therapeutics (available at the Nektar website) U.S. Pat. Nos. 4,640,835; 4,496,689; 4,301,144; 4,670,417; 4,791,192 or 4,179,337, all entirely incorporated by reference. In addition, as is known in the art, amino acid substitutions may be made in various positions within the antibody to facilitate the addition of polymers such as PEG. See for example, U.S. Publication No. 2005/0114037A1, entirely incorporated by reference.

Therapeutic Antibodies

As will be appreciated by those in the art, the present invention finds use in any number of current or future therapeutic antibodies.

Virtually any antigen may be targeted by the antibody compositions of the invention, including but not limited to proteins, subunits, domains, motifs, and/or epitopes belonging to the following list of target antigens, which includes both soluble factors such as cytokines and membrane-bound factors, including transmembrane receptors: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Ax1, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCl, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-1 (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxini, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-1, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpi, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mud), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta R1 (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFα, TNF-RI, TNF-RII, TNFRSF10A (TRAIL R1Apo-2, DR4), TNFRSF10B (TRAIL R2DR5, KILLER, TRICK-2A, TRICK-B), TNFRSF10C (TRAIL R3DcR1, LIT, TRID), TNFRSF10D (TRAIL R4DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1R), TNFRSF5 (CD40 p50), TNFRSF6 (Fas Apo-1, APT1, CD95), TNFRSF6B (DcR3M68, TR6), TNFRSF7 (CD27), TNFRSF8 (CD30), TNFRSF9 (4-1BB CD137, ILA), TNFRSF21 (DR6), TNFRSF22 (DcTRAIL R2TNFRH2), TNFRST23 (DcTRAIL R1 TNFRH1), TNFRSF25 (DR3Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-α Conectin, DIF, TNFSF2), TNFSF1B (TNF-b LTa, TNFSF1), TNFSF3 (LTb TNFC, p33), TNFSF4 (0x40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factors.

One skilled in the art will appreciate that the aforementioned list of targets refers not only to specific proteins and biomolecules, but the biochemical pathway or pathways that comprise them. For example, reference to CTLA-4 as a target antigen implies that the ligands and receptors that make up the T cell co-stimulatory pathway, including CTLA-4, B7-1, B7-2, CD28, and any other undiscovered ligands or receptors that bind these proteins, are also targets. Thus target as used herein refers not only to a specific biomolecule, but the set of proteins that interact with said target and the members of the biochemical pathway to which said target belongs. One skilled in the art will further appreciate that any of the aforementioned target antigens, the ligands or receptors that bind them, or other members of their corresponding biochemical pathway, may be operably linked to the Fc variants of the present invention in order to generate an Fc fusion. Thus for example, an Fc fusion that targets EGFR could be constructed by operably linking an Fc variant to EGF, TGF-b, or any other ligand, discovered or undiscovered, that binds EGFR. Accordingly, an Fc variant of the present invention could be operably linked to EGFR in order to generate an Fc fusion that binds EGF, TGF-b, or any other ligand, discovered or undiscovered, that binds EGFR. Thus virtually any polypeptide, whether a ligand, receptor, or some other protein or protein domain, including but not limited to the aforementioned targets and the proteins that compose their corresponding biochemical pathways, may be operably linked to the Fc variants of the present invention to develop an Fc fusion.

The choice of suitable antigen depends on the desired application. For anti-cancer treatment it is desirable to have a target whose expression is restricted to the cancerous cells. Some targets that have proven especially amenable to antibody therapy are those with signaling functions. Other therapeutic antibodies exert their effects by blocking signaling of the receptor by inhibiting the binding between a receptor and its cognate ligand. Another mechanism of action of therapeutic antibodies is to cause receptor down regulation. Other antibodies do not work by signaling through their target antigen. In some cases, antibodies directed against infectious disease agents are used.

In one embodiment, the Fc variants of the present invention are incorporated into an antibody against a cytokine. Alternatively, the Fc variants are fused or conjugated to a cytokine. By “cytokine” as used herein is meant a generic term for proteins released by one cell population that act on another cell as intercellular mediators. For example, as described in Penichet et al., 2001, J Immunol Methods 248:91-101, expressly incorporated by reference, cytokines may be fused to antibody to provide an array of desirable properties. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; C5a; and other polypeptide factors including LIF and kit ligand (KL). As used herein, the term cytokine includes proteins from natural sources or from recombinant cell culture, and biologically active equivalents of the native sequence cytokines.

Cytokines and soluble targets, such as TNF superfamily members, are preferred targets for use with the variants of the present invention. For example, anti-VEGF, anti-CTLA-4, and anti-TNF antibodies, or fragments thereof, are particularly good antibodies for the use of Fc variants that increase the FcRn binding. Therapeutics against these targets are frequently involved in the treatment of autoimmune diseases and require multiple injections over long time periods. Therefore, longer serum half-lives and less frequent treatments, brought about from the variants of the present invention, are particularly preferred.

A number of antibodies and Fc fusions that are approved for use, in clinical trials, or in development may benefit from the Fc variants of the present invention. These antibodies and Fc fusions are herein referred to as “clinical products and candidates”. Thus in a preferred embodiment, the Fc polypeptides of the present invention may find use in a range of clinical products and candidates. For example, a number of antibodies that target CD20 may benefit from the Fc polypeptides of the present invention. For example the Fc polypeptides of the present invention may find use in an antibody that is substantially similar to rituximab (Rituxan®, IDEC/Genentech/Roche) (see for example U.S. Pat. No. 5,736,137), a chimeric anti-CD20 antibody approved to treat Non-Hodgkin's lymphoma; HuMax-CD20, an anti-CD20 currently being developed by Genmab, an anti-CD20 antibody described in U.S. Pat. No. 5,500,362, AME-133 (Applied Molecular Evolution), hA20 (Immunomedics, Inc.), HumaLYM (Intracel), and PRO70769 (PCT/US2003/040426, entitled “Immunoglobulin Variants and Uses Thereof”). A number of antibodies that target members of the family of epidermal growth factor receptors, including EGFR (ErbB-1), Her2/neu (ErbB-2), Her3 (ErbB-3), Her4 (ErbB-4), may benefit from the Fc polypeptides of the present invention. For example the Fc polypeptides of the present invention may find use in an antibody that is substantially similar to trastuzumab (Herceptin®, Genentech) (see for example U.S. Pat. No. 5,677,171), a humanized anti-Her2/neu antibody approved to treat breast cancer; pertuzumab (rhuMab-2C4, Omnitarg™), currently being developed by Genentech; an anti-Her2 antibody described in U.S. Pat. No. 4,753,894; cetuximab (Erbitux®, Imclone) (U.S. Pat. No. 4,943,533; PCT WO 96/40210), a chimeric anti-EGFR antibody in clinical trials for a variety of cancers; ABX-EGF (U.S. Pat. No. 6,235,883), currently being developed by Abgenix-Immunex-Amgen; HuMax-EGFr (U.S. Ser. No. 10/172,317), currently being developed by Genmab; 425, EMD55900, EMD62000, and EMD72000 (Merck KGaA) (U.S. Pat. No. 5,558,864; Murthy et al. 1987, Arch Biochem Biophys. 252(2):549-60; Rodeck et al., 1987, J Cell Biochem. 35(4):315-20; Kettleborough et al., 1991, Protein Eng. 4(7):773-83); ICR62 (Institute of Cancer Research) (PCT WO 95/20045; Modjtahedi et al., 1993, J. Cell Biophys. 1993, 22(1-3):129-46; Modjtahedi et al., 1993, Br J. Cancer. 1993, 67(2):247-53; Modjtahedi et al, 1996, Br J Cancer, 73(2):228-35; Modjtahedi et al, 2003, Int J Cancer, 105(2):273-80); TheraCIM hR3 (YM Biosciences, Canada and Centro de Immunologia Molecular, Cuba (U.S. Pat. No. 5,891,996; U.S. Pat. No. 6,506,883; Mateo et al, 1997, Immunotechnology, 3(1):71-81); mAb-806 (Ludwig Institue for Cancer Research, Memorial Sloan-Kettering) (Jungbluth et al. 2003, Proc Natl Acad Sci USA. 100(2):639-44); KSB-102 (KS Biomedix); MR1-1 (IVAX, National Cancer Institute) (PCT WO 0162931A2); and SC100 (Scancell) (PCT WO 01/88138). In another preferred embodiment, the Fc polypeptides of the present invention may find use in alemtuzumab (Campath®, Millenium), a humanized monoclonal antibody currently approved for treatment of B-cell chronic lymphocytic leukemia. The Fc polypeptides of the present invention may find use in a variety of antibodies or Fc fusions that are substantially similar to other clinical products and candidates, including but not limited to muromonab-CD3 (Orthoclone OKT3®), an anti-CD3 antibody developed by Ortho Biotech/Johnson & Johnson, ibritumomab tiuxetan (Zevalin®), an anti-CD20 antibody developed by IDEC/Schering AG, gemtuzumab ozogamicin (Mylotarg®), an anti-CD33 (p67 protein) antibody developed by Celltech/Wyeth, alefacept (Amevive®), an anti-LFA-3 Fc fusion developed by Biogen), abciximab (ReoPro®), developed by Centocor/Lilly, basiliximab (Simulect®), developed by Novartis, palivizumab (Synagis®), developed by Medlmmune, infliximab (Remicade®), an anti-TNFalpha antibody developed by Centocor, adalimumab (Humira®), an anti-TNFalpha antibody developed by Abbott, Humicade™, an anti-TNFalpha antibody developed by Celltech, etanercept (Enbrel®), an anti-TNFalpha Fc fusion developed by Immunex/Amgen, ABX-CBL, an anti-CD147 antibody being developed by Abgenix, ABX-IL8, an anti-IL8 antibody being developed by Abgenix, ABX-MA1, an anti-MUC18 antibody being developed by Abgenix, Pemtumomab (R1549, 90Y-muHMFG1), an anti-MUC1 In development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegren® (natalizumab), an anti-alpha-4-beta-1 (VLA-4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-β2 antibody being developed by Cambridge Antibody Technology, J695, an anti-IL-12 antibody being developed by Cambridge Antibody Technology and Abbott, CAT-192, an anti-TGFβ1 antibody being developed by Cambridge Antibody Technology and Genzyme, CAT-213, an anti-Eotaxin1 antibody being developed by Cambridge Antibody Technology, LymphoStat-B™ an anti-Blys antibody being developed by Cambridge Antibody Technology and Human Genome Sciences Inc., TRAIL-R1mAb, an anti-TRAIL-R1 antibody being developed by Cambridge Antibody Technology and Human Genome Sciences, Inc., Avastin™ (bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech, Xolair™ (Omalizumab), an anti-IgE antibody being developed by Genentech, Raptiva™ (Efalizumab), an anti-CD11a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-IL15, an anti-IL15 antibody being developed by Genmab and Amgen, HuMax-Inflam, being developed by Genmab and Medarex, HuMax-Cancer, an anti-Heparanase I antibody being developed by Genmab and Medarex and Oxford GcoSciences, HuMax-Lymphoma, being developed by Genmab and Amgen, HuMax-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDEC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide™ (labetuzumab), an anti-carcinoembryonic antigen (CEA) antibody being developed by Immunomedics, LymphoCide™ (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, Osidem™ (IDM-1), and anti-Her2 antibody being developed by Medarex and Immuno-Designed Molecules, HuMax™-CD4, an anti-CD4 antibody being developed by Medarex and Genmab, HuMax-IL15, an anti-IL15 antibody being developed by Medarex and Genmab, CNTO 148, an anti-TNFα antibody being developed by Medarex and Centocor/J&J, CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumab), an anti-CD3 antibody being developed by Protein Design Labs, HuZAF™, an anti-gamma interferon antibody being developed by Protein Design Labs, Anti-α5β1 Integrin, being developed by Protein Design Labs, anti-IL-12, being developed by Protein Design Labs, ING-1, an anti-Ep-CAM antibody being developed by Xoma, and MLN01, an anti-Beta2 integrin antibody being developed by Xoma, all of the above-cited references in this paragraph are expressly incorporated herein by reference.

Variant Fc Domains

The compositions of the invention comprise a variant Fc domain (either as part of the variant therapeutic antibody or as a fusion partner in a fusion composition, described herein). By “variant protein” or “protein variant”, or “variant” as used herein is meant a protein that differs from that of a parent protein by virtue of at least one amino acid modification, including insertions, deletions and substitutions, with the latter finding particular use in the present invention. Protein variant may refer to the protein itself, a composition comprising the protein, or the amino sequence that encodes it. Preferably, the protein variant has at least one amino acid modification compared to the parent protein, e.g. from about one to about seventy amino acid modifications, and preferably from about one to about five amino acid modifications compared to the parent. The protein variant sequence herein will preferably possess at least about 80% identity with a parent protein sequence, and most preferably at least about 90% identity, more preferably at least about 95% identity. Variant protein can refer to the variant protein itself, compositions comprising the protein variant, or the DNA sequence that encodes it. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising a modification in an Fc domain. The Fc variant domains of the present invention are defined according to the amino acid modifications that compose them. Thus, for example, S267E or 267E is an Fc variant with the substitution of a glutamic acid at position 267 relative to the parent Fc polypeptide, wherein the numbering is according to the EU index. Likewise, 267E/328F defines an Fc variant with the substitutions S267E and L328F. The identity of the WT amino acid may be unspecified, in which case the aforementioned variant is referred to as 267E/328F. It is noted that the order in which substitutions are provided is arbitrary, that is to say that, for example, 267E/328F is the same Fc variant as 328F/267E, and so on. For all positions discussed in the present invention, numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids (which generally finds the most use in the present invention when the compositions of the invention are produced recombinantly) and non-naturally occurring amino acids. Variants may comprise non-natural amino acids. Examples include U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

As used herein, “protein” herein is meant at least two covalently attached amino acids, which includes proteins, polypeptides, oligopeptides and peptides. The peptidyl group may comprise naturally occurring amino acids and peptide bonds, or synthetic peptidomimetic structures, i.e. “analogs”, such as peptoids (see Simon et al., PNAS USA 89(20):9367 (1992), entirely incorporated by reference). The amino acids may either be naturally occurring or non-naturally occurring; as will be appreciated by those in the art, again with naturally occurring amino acids being preferred when the compositions of the invention are produced recombinantly. For example, homo-phenylalanine, citrulline, and noreleucine are considered amino acids for the purposes of the invention, and both D- and L- (R or S) configured amino acids may be utilized. The variants of the present invention may comprise modifications that include the use of unnatural amino acids incorporated using, for example, the technologies developed by Schultz and colleagues, including but not limited to methods described by Cropp & Shultz, 2004, Trends Genet. 20(12):625-30, Anderson et al., 2004, Proc Natl Acad Sci USA 101(2):7566-71, Zhang et al., 2003, 303(5656):371-3, and Chin et al., 2003, Science 301(5635):964-7, all entirely incorporated by reference. In addition, polypeptides may include synthetic derivatization of one or more side chains or termini, glycosylation, PEGylation, circular permutation, cyclization, linkers to other molecules, fusion to proteins or protein domains, and addition of peptide tags or labels, some of which are described below.

Specific Amino Acid Substitutions for Variant Fc Domains

There are a wide variety of amino acid substitutions within the Fc domain that find use in the present invention, as described herein. In some embodiments, any amino acid substitutions that increase the binding affinity of the variant Fc domain to the FcγRIIb receptor (often referred to herein as “IIb variants”). As is known, the K_{D} for the human wild-type IgG1 Fc domain is generally in the μM range, as shown below. In the embodiments of the invention, the K_{D} for the variant Fc domain is generally in the nM range, with K_{D}s of less than about 100 nM finding particular use in some embodiments. That is, the affinity of the Fc variant domain has a K_{D} less than about 100 nM, e.g., less than or equal to about 95 nM, less than or equal to about 90 nM, less than or equal to about 85 nM, less than or equal to about 80 nM, less than or equal to about 75 nM, less than or equal to about 74 nM.

Alternatively, optionally or in addition, the binding affinity of the variant Fc domain to the FcγRIIb receptor can be at least about 50 fold, 100 fold, 150 fold, 200 fold, 300 fold, 400 fold or higher greater than the affinity of a human wild type Fc domain, such as those from IgG1 (although other wild type Fc domains are also included).

In addition, there are amino acid substitutions that surprisingly increase binding to several FcγR receptors, including FcγRIIb as well as FcγRIIIa, for example. Alternatively, there are amino acid substitutions that act mostly or solely on the FcγRIIb receptor.

Substitutions to enhance FcγR affinity, in particular to FcγRIIb, include substitutions made at one or more of Fc positions selected from the group consisting of 234, 235, 236, 237, 239, 266, 267, 268, 325, 326, 327, 328, and 332, wherein numbering is according to the EU index. In this embodiment, as well as for all such lists, amino acid substitutions can be selected independently and optionally independently combined with any other amino acid substitution.

In particular, substitutions are made to at least one or more of the nonlimiting following positions to enhance affinity to FcγRIIb: 235, 236, 239, 266, 267, 268, and 328.

Substitutions for enhancing affinity to FcγRIIb include but are not limited to: 234D, 234E, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E, wherein numbering is according to the EU index. More preferred substitutions for enhancing affinity to FcγRIIb include but are not limited to: 235Y, 236D, 239D, 266M, 267E, 268D, 268E, 328F, 328W, and 328Y.

Combinations of substitutions for enhancing affinity to FcγRIIb include: 234D/267E, 234E/267E, 234F/267E, 234E/328F, 234W/239D, 234W/239E, 234W/267E, 234W/328Y, 235D/267E, 235D/328F, 235F/239D, 235F/267E, 235F/328Y, 235Y/236D, 235Y/239D, 235Y/267D, 235Y/267E, 235Y/268E, 235Y/328F, 236D/239D, 236D/267E, 236D/268E, 236D/328F, 236N/267E, 237D/267E, 237N/267E, 239D/267D, 239D/267E, 239D/268D, 239D/268E, 239D/327D, 239D/328F, 239D/328W, 239D/328Y, 239D/332E, 239E/267E, 266M/267E, 267D/268E, 267E/268D, 267E/268E, 267E/325L, 267E/327D, 267E/327E, 267E/328F, 267E/3281, 267E/328Y, 267E/332E, 268D/327D, 268D/328F, 268D/328W, 268D/328Y, 268D/332E, 268E/328F, 268E/328Y, 327D/328Y, 328F/1332E, 328W/332E, and 328Y/332E, wherein numbering is according to the EU index.

In some embodiments, combinations of substitutions for enhancing affinity to FcγRIIb include, but are not limited to: 235Y/267E, 236D/267E, 239D/268D, 239D/267E, 267E/268D, 267E/268E, and 267E/328F.

Substitutions or combinations of substitutions for enhancing affinity to FcγRIIb may include but are not limited to: 234F/236N, 234F/236D, 236A/237A, 236S/237A, 235D/239D, 234D/267E, 234E/267E, 234F/267E, 235D/267E, 235F/267E, 235S/267E, 235T/267E, 235Y/267D, 235Y/267E, 236D/267E, 236E/267E, 236N/267E, 237D/267E, 237N/267E, 239D/267D, 239D/267E, 266M/267E, 234E/268D, 236D/268D, 239D/268D, 267D/268D, 267D/268E, 267E/268D, 267E/268E, 267E/325L, 267D/327D, 267D/327E, 267E/327D, 267E/327E, 268D/327D, 239D/328Y, 267E/328F, 267E/328H, 267E/3281, 267E/328Q, 267E/328Y, 268D/328Y, 239D/332E, 328Y/332E, 234D/236N/267E, 235Y/236D/267E, 234W/239E/267E, 235Y/239D/267E, 236D/239D/267E, 235Y/267E/268E, 236D/267E/268E, 239D/267E/268E, 234W/239D/328Y, 235F/239D/328Y, 234E/267E/328F, 235D/267E/328F, 235Y/267E/328F, 236D/267E/328F, 239D/267A/328Y, 239D/267E/328F, 234W/268D/328Y, 235F/268D/328Y, 239D/268D/328F, 239D/268D/328W, 239D/268D/328Y, 239D/268E/328Y, 267A/268D/328Y, 267E/268E/328F, 239D/326D/328Y, 268D/326D/328Y, 239D/327D/328Y, 268D/327D/328Y, 239D/267E/332E, 234W/328Y/332E, 235F/328Y/332E, 239D/328F/332E, 239D/328Y/332E, 267A/328Y/332E, 268D/328F/332E, 268D/328W/332E, 268D/328Y/332E, 268E/328Y/332E, 326D/328Y/332E, 327D/328Y/332E, 234W/236D/239E/267E, 239D/268D/328F/332E, 239D/268D/328W/332E, and 239D/268D/328Y/332E, wherein numbering is according to an EU index. Substitutions or combinations of substitutions for enhancing affinity to FcγRIIb may include but are not limited to: 266D, 234F/236N, 234F/236D, 236A/237A, 236S/237A, 235D/239D, 234D/267E, 234E/267E, 234F/267E, 235D/267E, 235F/267E, 235S/267E, 235T/267E, 235Y/267D, 236D/267E, 236E/267E, 236N/267E, 237D/267E, 237N/267E, 266M/267E, 234E/268D, 236D/268D, 267D/268D, 267D/268E, 267E/268D, 267E/268E, 267E/325L, 267D/327D, 267D/327E, 267E/327E, 268D/327D, 239D/328Y, 267E/328F, 267E/328H, 267E/3281, 267E/328Q, 267E/328Y, 268D/328Y, 234D/236N/267E, 235Y/236D/267E, 234W/239E/267E, 235Y/239D/267E, 236D/239D/267E, 235Y/267E/268E, 236D/267E/268E, 234W/239D/328Y, 235F/239D/328Y, 234E/267E/328F, 235D/267E/328F, 235Y/267E/328F, 236D/267E/328F, 239D/267A/328Y, 239D/267E/328F, 234W/268D/328Y, 235F/268D/328Y, 239D/268D/328F, 239D/268D/328W, 239D/268D/328Y, 239D/268E/328Y, 267A/268D/328Y, 267E/268E/328F, 239D/326D/328Y, 268D/326D/328Y, 239D/327D/328Y, 268D/327D/328Y, 234W/328Y/332E, 235F/328Y/332E, 239D/328F/332E, 239D/328Y/332E, 267A/328Y/332E, 268D/328F/332E, 268D/328W/332E, 268D/328Y/332E, 268E/328Y/332E, 326D/328Y/332E, 327D/328Y/332E, 234W/236D/239E/267E, 239D/268D/328F/332E, 239D/268D/328W/332E, and 239D/268D/328Y/332E, wherein numbering is according to an EU index.

Substitutions or combinations of substitutions for enhancing affinity to FcγRIIb may include but are not limited to: 234N, 235Q, 235R, 235W, 235Y, 236D, 236H, 2361, 236L, 236S, 236Y, 237H, 237L, 239D, 239N, 2661, 266M, 267A, 267D, 267E, 267G, 268D, 268E, 268N, 268Q, 298E, 298L, 298M, 298Q, 326A, 326E, 326W, 327D, 327L, 328E, 328F, 330D, 330H, 330K, 234F/236N, 234F/236D, 235D/239D, 234D/267E, 234E/267E, 234F/267E, 235D/267E, 235F/267E, 235T/267E, 235Y/267D, 235Y/267E, 236D/267E, 236E/267E, 236N/267E, 237D/267E, 237N/267E, 239D/267D, 239D/267E, 266M/267E, 234E/268D, 236D/268D, 239D/268D, 267D/268D, 267D/268E, 267E/268D, 267E/268E, 267E/325L, 267D/327D, 267D/327E, 267E/327D, 267E/327E, 268D/327D, 239D/328Y, 267E/328F, 267E/328H, 267E/3281, 267E/328Q, 267E/328Y, 268D/328Y, 239D/332E, 328Y/332E, 234D/236N/267E, 235Y/236D/267E, 234W/239E/267E, 235Y/239D/267E, 236D/239D/267E, 235Y/267E/268E, 236D/267E/268E, 239D/267E/268E, 234W/239D/328Y, 235F/239D/328Y, 234E/267E/328F, 235D/267E/328F, 235Y/267E/328F, 236D/267E/328F, 239D/267A/328Y, 239D/267E/328F, 234W/268D/328Y, 235F/268D/328Y, 239D/268D/328F, 239D/268D/328W, 239D/268D/328Y, 239D/268E/328Y, 267A/268D/328Y, 267E/268E/328F, 239D/326D/328Y, 268D/326D/328Y, 239D/327D/328Y, 268D/327D/328Y, 239D/267E/332E, 234W/328Y/332E, 235F/328Y/332E, 239D/328F/332E, 239D/328Y/332E, 267A/328Y/332E, 268D/328F/332E, 268D/328W/332E, 268D/328Y/332E, 268E/328Y/332E, 326D/328Y/332E, 327D/328Y/332E, 234W/236D/239E/267E, 239D/268D/328F/332E, 239D/268D/328W/332E, and 239D/268D/328Y/332E. Again, as for all such lists, individual variants, including combination variants, can independently be included or excluded from each list.

In addition, it should be noted that additional substitutions can be made in the Fc region, as is generally described in U.S. Ser. Nos. 12/341,769, 10/672,280, 10/822,231, 11/124,620 and 11/396,495, all of which are incorporated herein expressly in their entirety, and particularly for the disclosure of particular substitutions and associated binding data. In particular, amino acid substitutions that result in increased binding to the FcRn receptor and/or increased in vivo half life of Fc domains containing such variants are included, including, but not limited to, 308FCYW, 2591, 428L, 434S, 2591/308F, 428L/4345, 2591/308F/428L and 308F/428L. Similarly, if increased ADCC (increased binding to FcγRIIIa) is desired, amino acid substitutions include, but are not limited to, 239D/332E and others shown in FIG. 41 of Ser. No. 11/124,620.

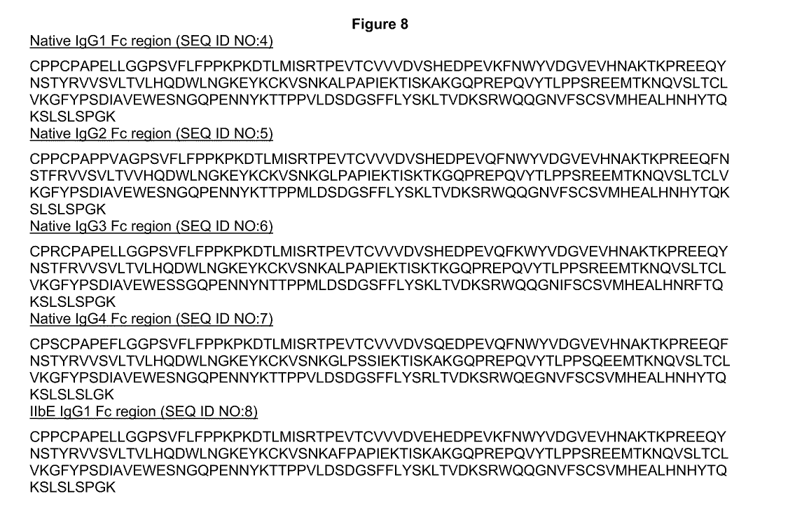

Variants can be constructed in the Fc region of any antibody or biotherapeutic Fc fusion (e.g. fusion composition). FIG. 8 provides amino acid sequences of the Fc regions of the native human IgG isotypes, as well as the Fc-engineered 267E/328F IIbE IgG1 version described in the Examples.

The use of the particular 267E/328F variant is meant here as proof of concept for the mechanism as described herein, and is not meant to constrain the invention to their particular use. The data provided in U.S. Ser. No. 12/156,183 and U.S. Ser. No. 11/124,620 (both of which are expressly incorporated herein by reference) indicate that a number of engineered variants, at specific Fc positions, provide the targeted properties.

In addition the incorporation of the variant Fc domains of the invention into therapeutic antibodies, the variant Fc domains may also be linked to therapeutic proteins (which are not antibodies), autoantigens and/or allergens to reduce the immunogenicity of these immunogens.

Therapeutic Proteins

Therapeutic proteins, sometimes also referred to as “biologic drugs” that may be variant Fc domain fusion partners for the immunoprotective effect of the variant Fc regions of the invention include but are not limited to: recombinant human growth hormone, gonadotropin-releasing hormone, human chorionic gonadotropin, salmon calcitonin, recombinant human erythropoietin, insulin, GnRH, edenileukin diftitox,adenosine deamidase, megakaryocyte-derived growth factor (MGDF, thrombopoietin), glucocerebrosidase, Alpha-galactosidase, tissue plasminogen activator, Glucagon-like peptide-1 (GLP-1), and urokinase, enzymes including Factor VIII, Factor VIIa, rhDNase, recombinant tissue plasminogen activator, recombinant streptokinase, recombinant staphylokinase, recombinant cytokines including IFN-alpha 2a and IFN-alpha 2b, IFN-beta Ia and Ib, IL-2, IL-3, CTNF, growth factors including IL-3, GM-CSF fusion protein (e.g., PIXY321), GM-CSF, and human G-CSF (Schellekens 2002, Clinical therapeutics 24[11]:1720-40; discussion 1719; Shankar et al., 2006, Trends in biotechnology 24[6]:274-80). Preferred biologics include those dosed chronically and those known to be immunogenic in clinical use, with examples including Factor VIII, Factor VII, salmon calcitonin, erythropoietin, lenercept, and human growth hormone.

Autoantigens and Allergens

The present invention finds use in the “tolerization” of immunogens, such that undesirable B-cell mediated immune responses are reduced or eliminated. This approach, outlined in FIG. 9, mimics the inhibitory effects of immune complex by high-affinity coengagement of Fc RIIb and the BCR coreceptor complex on human B cells. A key step in immune response to an autoantigen or allergen is engagement with specific BCR on B cells followed by activation, internalization, and presentation to T cells. Fusion of the autoantigen or allergen to the IgG Fc region enables interaction with the inhibitory receptor FcγRIIb. However, because native IgG's, for example IgG1, bind FcγRIIb with weak (uM) affinity, FcγRIIb-mediated inhibition occurs in response only to immune complexed but not monomeric IgG Fc.

Thus this strategy is also based on generating a high affinity interaction between the Fc region and FcγRIIb, which may enable maximal inhibition of B cell activation by monovalent (non-immune complexed) autoantigen or allergen. Coupling of FcγRIIb-enhanced Fc domains to autoantigen or allergen utilizes the natural inhibitory pathway to inhibit the B cell response to the fusion partner. Enhanced affinity of the protein-Fc fusion for the inhibitory FcγRIIb prevents (e.g. reduces) anti-autoantigen or anti-allergen B cells from activation and differentiation into immunoglobulin-producing plasma cells.

In some embodiments, the immunogen is an allergen. By “allergen” herein is meant a substance that produces an inflammatory and/or allergic reaction in some population of patients. Allergens are generally exogeneous to the host or patient; that is, the allergenic substance is not normally found within the host or patient. In some embodiments, the immunogen is an autoantigen. By “autoantigen” herein is meant a substance, generally endogeneous to the host (patient), that results in undesirable immune reactions. Autoantigens are generally endogeneous to the host or patient; that is, they are found within the patient but the patient is displaying an undesirable immune response to the autoantigen.

Autoimmune diseases that may be treated by this approach include allogenic islet graft rejection, alopecia greata, ankylosing spondylitis, antiphospholipid syndrome, autoimmune Addison's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune disfunction syndrome, chronic inflammatory demyelinating polyneuropathy, Churg-Strauss syndrome, cicatrical pemphigoid, CREST syndrome, cold agglutinin disease, Crohn's disease, dermatomyositis, discoid lupus, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen plantus, lupus erthematosis, Meniere's disease, mixed connective tissue disease, multiple sclerosis, type 1 diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobinulinemia, primary biliary cirrhosis, psoriasis, psoriatic arthritis, Reynauld's phenomenon, Reiter's syndrome, rheumatoid arthritis, sarcoidosis, scleroderma, Sjorgen's syndrome, solid organ transplant rejection, stiff-man syndrome, systemic lupus erythematosus, takayasu arteritis, temporal arteristis/giant cell arteritis, thrombotic thrombocytopenia purpura, ulcerative colitis, uveitis, vasculitides such as dermatitis herpetiformis vasculitis, vitiligo, and Wegner's granulomatosis. Thus, any molecule associated with the above disorders may be used as the immunogen to which an immunoprotective Fc domain is fused as described herein.

Inflammatory and allergic disorders that may be treated by this approach include acute respiratory distress syndrome (ARDS), acute septic arthritis, adjuvant arthritis, juvenile idiopathic arthritis, allergic encephalomyelitis, allergic rhinitis, allergic vasculitis, allergy, asthma, atherosclerosis, chronic inflammation due to chronic bacterial or viral infectionis, chronic obstructive pulmonary disease (COPD), coronary artery disease, encephalitis, inflammatory bowel disease, inflammatory osteolysis, inflammation associated with acute and delayed hypersensitivity reactions, inflammation associated with tumors, peripheral nerve injury or demyelinating diseases, inflammation associated with tissue trauma such as burns and ischemia, inflammation due to meningitis, multiple organ injury syndrome, pulmonary fibrosis, sepsis and septic shock, Stevens-Johnson syndrome, undifferentiated arthropy, and undifferentiated spondyloarthropathy. Thus, any molecule associated with the above disorders may be used as the immunogen to which an immunoprotective Fc domain is fused as described herein.

By fusing immunoprotective Fc domains to the autoimmune antigens or allergens that play a role in these disorders, such biotherapeutics may mimic the suppressive effects of cognate immune complex on activated B cells. Because FcγRIIb coengagement inhibits BCR-dependent antigen internalization and processing, the activities of the IIbE variants presented here may also suppress T cell-mediated adaptive immunity, thereby inhibiting the underlying biology of the disease.

Autoimmune antigens and allergens that may be Fc fusions partners for the immunoprotective Fc regions of the invention include but are not limited to double-stranded DNA, platelet antigens, myelin protein antigen, Sm antigens in snRNPs, islet cell antigen, Rheumatoid factor, and anticitrullinated protein. citrullinated proteins and peptides such as CCP-1, CCP-2 (cyclical citrullinated peptides), fibrinogen, fibrin, vimentin, fillaggrin, collagen I and II peptides, alpha-enolase, translation initiation factor 4G1, perinuclear factor, keratin, Sa (cytoskeletal protein vimentin), components of articular cartilage such as collagen II, IX, and XI, circulating serum proteins such as RFs (IgG, IgM), fibrinogen, plasminogen, ferritin, nuclear components such as RA33/hnRNP A2, Sm, eukaryotic trasnlation elogation factor 1 alpha 1, stress proteins such as HSP-65, -70, -90, BiP, inflammatory/immune factors such as B7-H1, IL-1 alpha, and IL-8, enzymes such as calpastatin, alpha-enolase, aldolase-A, dipeptidyl peptidase, osteopontin, glucose-6-phosphate isomerase, receptors such as lipocortin 1, neutrophil nuclear proteins such as lactoferrin and 25-35 kD nuclear protein, granular proteins such as bactericidal permeability increasing protein (BPI), elastase, cathepsin G, myeloperoxidase, proteinase 3, platelet antigens, myelin protein antigen, islet cell antigen, rheumatoid factor, histones, ribosomal P proteins, cardiolipin, vimentin, nucleic acids such as dsDNA, ssDNA, and RNA, ribonuclear particles and proteins such as Sm antigens (including but not limited to SmD's and SmB'/B), U1 RNP, A2/B1 hnRNP, Ro (SSA), La (SSB) antigens, Derp1, B pollen, and ragweed.

Autoantigens common in many autoimmune diseases including rheumatoid arthritis and SLE that may be Fc fusions partners for the immunoprotective Fc regions of the invention include but are not limited to (SwissProt references are in parentheses): SmB/SmB′ (P14678), Sm-D1 (P62314), Sm-D2 (P62316), Sm-D3 (P62318), U1 snRNP A (P09012), U1 snRNP 70K (P08621), U1 snRNP C(P09234), U2 snRNP A′ (P09661), U2 snRNP B″ (P08579), Ro52K SS-A1 (P19474), Ro60K SS-A2 (P10155), La SS-B (P05455), Histone H1b (P10412), Histone H2A.1b (P02261), Histone H2B.1a (P62807), Histone H3.1 (P16106), Histone H4 (P62805), DNA topoisomerase I (P11387), CENP-A (P49450), CENP-B (P07199), CENP-C (003188), Ku86 (P13010), Ku70 (P12956), Annexin A11 (P50995), RNaseP p38 (P78345), RNaseP p30 (P78346), RuvB-like 1 (Q9Y265), CHD-3 (012873), CHD-4 (014839), RCC1 (P18754), PM/Scl-100, PM/Scl-2 (001780), PM/Scl-75, PM/Scl-1 (006265), RRP42 (015024), RRP4 (013868), Fibrillarin (P22087), UBF-1 (P17480), PA28g (P61289), SSNA1 (043805), hnRNP NB (099729), hnRNP A2 (P22626), ZNF330 (Q9Y3S2), ASF-1/SRp30a (007955), SC35 SRp30b (001130), SRp20 (P84103), SRp75 (008170), SRp40 (013243), SRp55 (013247), DBP1 (P67809), NUMA1 (014980), Eg5Kinesin-like NUMA-2 (P52732), PCNA (cyclin) (P12004) (Ndhlovu et al., 2011, Brain, behavior, and immunity 25[2]:279-285; Riemekasten et al., 2005, Rheumatology 44[8]:975-82; Goeb et al., 2009, Arthritis research & therapy 11[2]:R38; Auger et al., 2009, Annals of the rheumatic diseases 68[4]:591-4; Tilleman et al., 2007, Proteomics. Clinical applications 1[1]:32-46; Matsuo et al., 2006, Arthritis research & therapy 8[6]:R175; Corrigall et al., 2002, Critical reviews in immunology 22[4]:281-93; Carl et al., 2005, Arthritis research & therapy 7[6]:R1360-74; Chen et al., 2005, Autoimmunity reviews 4[3]:117-22; Schmitt 2003, Biomedicine & pharmacotherapy=Biomedecine & pharmacotherapie 57[7]:261-8; Rosen et al., 2009, Journal of internal medicine 265[6]:625-31; Graham et al., 2005, Current opinion in rheumatology 17[5]:513-7).

As specific autoantigens relevant to SLE, with or without central nervous system involvement, the following are example Fc fusion partners for the immunoprotective Fc regions: peroxiredoxin-4, ubiquitin carboxyl-terminal hydrolase isozyme L1, splicing factor arginine/serine-rich 3, histone H2A type 1, histone 2A, SC35, U1-70 k, SmB/B′, PML, topoisomerase I, CENP-B, CENP-C, fibrillarin, UBF, SmD, SmBO/B, ANA, dsDNA, U1 RNP, Sm, Ro, La, ribosomal P, cardiolipin, ssDNA, and ribosomal P (Riemekasten et al., 2005, Rheumatology 44[8]:975-82; lizuka et al., 2010, Lupus 19[6]:717-26).

As specific autoantigens relevant to MS, the following are example Fc fusion partners for the immunoprotective Fc regions: myelin basic protein (MBP), myelin oligodendrocyte glycoprotein (MOG), myelin associated glycoprotein (MAG), proteolipid protein (PLP), cyclic nucleotide phosphodiesterase (CNP), and neurofascin (Schmidt 1999, Multiple sclerosis 5[3]:147-60).

As specific autoantigens relevant to Type I Diabetes, the following are example Fc fusion partners for the immunoprotective Fc regions: insulin/proinsulin, glutamic acid decarboxylase (GAD), protein tyrosine phosphatase-like proteins IA-2 and IA-2β (Christie 1996, European journal of clinical investigation 26[10]:827-38).

As a specific autoantigen relevant to myasthenia gravis, the following is an example Fc fusion partner for the immunoprotective Fc regions: acetylcholine receptor (Sheng et al., 2009, Muscle & nerve 40[2]:279-86).

As a specific autoantigen relevant to Grave's disease, the following is an example Fc fusion partner for the immunoprotective Fc regions: thyroid stimulating hormone receptor (Chen et al., 2003, The Journal of clinical investigation 111[12]:1897-904).

Examples of common allergen proteins implicated in allergy and asthma (Aalberse 2000, The Journal of allergy and clinical immunology 106[2]:228-38) that may be Fc fusion partners for the immunoprotective Fc regions of the invention include but are not limited to (PDB references to structures or homology models are in parentheses): Grass group 2 (1BMW, 1WHO, 1WHP), Grass group 1, Grass group 3, Mite group 2 (1A9V, 1AHK, 1AHM), Serine proteases (example: 1DPO, trypsin), Mite group 3, Mite group 6, Mite group 9, Soybean Kunitz-type trypsin inhibitor (1AVW), Ole e 1, Grass group 11, Fruits group 2: thaumatin (1AUN), Vicilin: peanut Ara h 1 (1CAW, 1DGR, 1DGW), Tree group 1 (1BTV, 1BV1), Lipocalin, Milk 3-lactoglobulin (1BLG), Mouse (1MUP) and rat urinary protein, (2A2G, 2A2U), Dog Can f 1, Dog Can f 2, Bovine Bos d 1, Horse Equ c 1 (1BJ7), Cockroach Bla g 4, Cystatin: cat allergen 430 (1A67, 1CEW), Profilin (1CQA), Aspartate protease (2REN), Cockroach Bla g 2, Mite group 1 (2ACT, 1CSB), Lysozyme (1HEL)/lactalbumin (1HFZ), Vespid group 5 (1CFE), Ovotransferrin=conalbumin (10VT), Cyclophilin (2CYH), Grass group 4, Tree group 7, Phospholipase A2 (1POC), Nonspecific lipid transfer protein (1BWO), Seed 2S albumin (1PNB), Insect hemoglobin (1 ECO), Fish parvalbumin (1CPD, 5CPV), Calmodulin (10SA), Bet v 4, Jun o 2, Phl p 7, Mellitin from bee venom (1MLT), Fel d 1 chain 1 (2UTG), Serum albumin (1UOR), pectate lyase (1AIR, 2PEC), Amb e 1, Amb e 2, Cry j 1, Serine protease inhibitor (Serpin-family), Ovalbumin (10VA), PLA1 1LPA, Glutathione S-transferase (1HNB, 1GTA), Cockroach group 5, Mite group 8, Schistosomal glutathione S-transferase, Mitogillin: Asp f 1 (1AQ2), MnSOD Asp f 6 (1MNG), Enolase (1NEL), Amylase (1JAE), Ovotransferrin (1OVT), Coiled coil: tropomyosin (1C1G, 1TMZ, 2TMA), Shrimp group 1, Mite group 10, Ovomucoid (third domain only) 10MU, 1OVO, 1CT4, Hevein 1HEV, Amb e 5 1BBG, 2BBG, 3BBG (Aalberse 2000, The Journal of allergy and clinical immunology 106[2]:228-38).

Some preferred common environmental allergens for incorporation of the immunoprotective Fc regions are: Bla g 1 (cockroach), Can f 1 (dog), Der f 1 (Dermatophagoides farinae), Der p 1 (Dermatophagoides pteronyssinus), Fel d 1 (cat), Alt A1 and A1t A2 (Alternaria alternata), and MUP (mouse) (Arbes et al., 2005, The Journal of allergy and clinical immunology 116[2]:377-83; Salo et al., 2008, The Journal of allergy and clinical immunology 121[3]:678-684 e2).

Some preferred food allergens that may be Fc fusion partners for the immunoprotective Fc regions of the invention include but are not limited to: major fish allergen parvalbumin, the cow's milk allergens casein and βLg, peanut allergens including Ara h1, Ara h2, and Ara h3, and the nsLTPs and Bet v 1 homologs found in a variety of plant foods (Breiteneder et al., 2005, The Journal of allergy and clinical immunology 115[1]:14-23; quiz 24).

An exemplary autoantigen for use in the immunoprotection mechanism of the invention is myelin oligodendrocyte glycoprotein (MOG). MOG is a glycoprotein of the myelin sheath that has been intensively studied as an autoantigen in demyelinating diseases such as multiple sclerosis (MS) (Lalive, 2008, Swiss Med Wkly 138[47-48]:692-707). As shown in the examples, MOG may be coupled to a variant Fc domain as outlined herein to reduce immunogenicity and thus treat the disease, in this case MS.

Fc Fusion Compositions

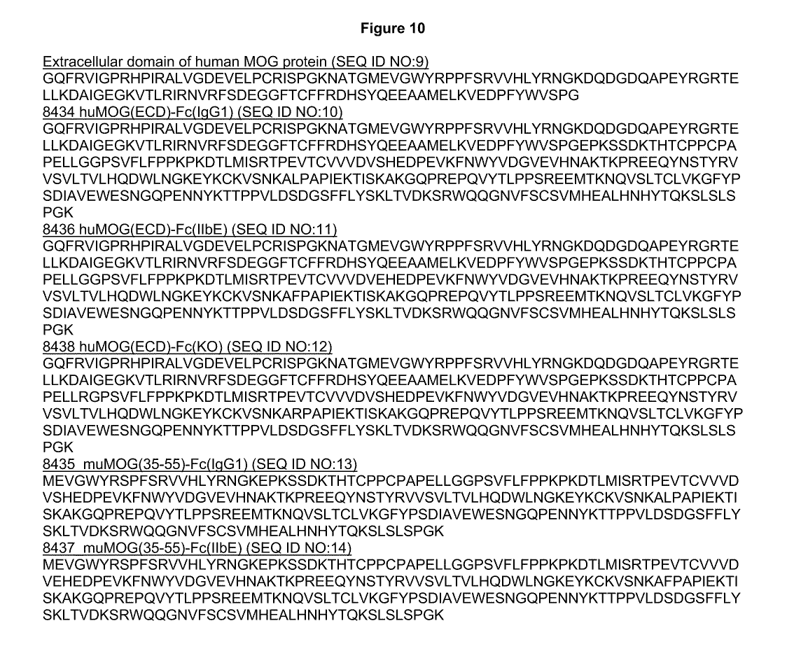

In the case of immunogens that are autoantigens or allergens, the present invention provides compositions comprising a first fusion protein comprising the autoantigen or allergen and a second fusion protein comprising a variant Fc domain as described above. The terms “first” and “second” are not meant to confer an orientation of the sequences with respect to the N- and C-terminal orientation of the two components. For example, the immunogen can be connected at its C-terminus to the N-terminus of the variant Fc domain, or at its N-terminus to the C-terminus of the variant Fc domain. The former is the configuration of the constructs shown in FIG. 10. In addition, the fusion partners may be linked directly or indirectly through the use of a linker. A direct linkage is generally where the N- and C-termini are covalently attached, and are generally constructed by aligning the coding regions of the first and second domains into a single nucleic acid that is expressed, as is more fully outlined herein.

Alternatively, a variety of linkers may find use in the present invention to covalently link Fc variant domains to the fusion partner (e.g. the immunogen) to form a fusion composition. By “linker “,” linker sequence”, “spacer”, “tethering sequence” or grammatical equivalents thereof, herein is meant a molecule or group of molecules (such as a monomer or polymer) that connects two molecules and often serves to place the two molecules in a preferred configuration. A number of strategies may be used to covalently link molecules together. These include, but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. Choosing a suitable linker for a specific case where two polypeptide chains are to be connected depends on various parameters, including but not limited to the nature of the two polypeptide chains (e.g., whether they naturally oligomerize), the distance between the N- and the C-termini to be connected if known, and/or the stability of the linker towards proteolysis and oxidation. Furthermore, the linker may contain amino acid residues that provide flexibility. Thus, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. Suitable lengths for this purpose include at least one and not more than 50 amino acid residues. Preferably, the linker is from about 1 to 30 amino acids in length, with linkers of 1 to 20 amino acids in length being most preferred. In addition, the amino acid residues selected for inclusion in the linker peptide should exhibit properties that do not interfere significantly with the activity of the other fusion domains. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains. Useful linkers include glycine-serine polymers (including, for example, (GS)n, (GSGGS)n SEQ ID NO: 15, (GGGGS)n SEQ ID NO:16, and (GGGS)n SEQ ID NO:17, where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies. Suitable linkers may also be identified by screening databases of known three-dimensional structures for naturally occurring motifs that can bridge the gap between two polypeptide chains. In a preferred embodiment, the linker is not immunogenic when administered in a human patient. Thus linkers may be chosen such that they have low immunogenicity or are thought to have low immunogenicity. For example, a linker may be chosen that exists naturally in a human. In a most preferred embodiment, the linker has the sequence of the hinge region of an antibody, that is the sequence that links the antibody Fab and Fc regions; alternatively the linker has a sequence that comprises part of the hinge region, or a sequence that is substantially similar to the hinge region of an antibody. Another way of obtaining a suitable linker is by optimizing a simple linker, e.g., (Gly4Ser)n SEQ ID NO:18, through random mutagenesis. Alternatively, once a suitable polypeptide linker is defined, additional linker polypeptides can be created to select amino acids that more optimally interact with the domains being linked. Other types of linkers that may be used in the present invention include artificial polypeptide linkers and inteins. In another embodiment, disulfide bonds are designed to link the two molecules. In another embodiment, linkers are chemical cross-linking agents. For example, a variety of bifunctional protein coupling agents may be used, including but not limited to N-succinimidyl-3-(2-pyridyldithiol) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., 1971, Science 238:1098. Chemical linkers may enable chelation of an isotope. For example, Carbon-14-labeled 1-isothiocyanatobenzyl-3-methyldiethylene triaminepentaacetic acid (MX-DTPA) is an exemplary chelating agent for conjugation of radionucleotide to the antibody (see PCT WO 94/11026). The linker may be cleavable, facilitating release of the cytotoxic drug in the cell. For example, an acid-labile linker, peptidase-sensitive linker, dimethyl linker or disulfide-containing linker (Chari et al., 1992, Cancer Research 52: 127-131) may be used. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers, that is may find use to link the Fc variants of the present invention to a fusion or conjugate partner to generate an Fc fusion, or to link the Fc variants of the present invention to a conjugate.

Methods of Making the Compositions of the Invention

The present invention provides methods of making the compositions of the invention. In general, the compositions of the invention, including therapeutic antibodies with variant Fc domains and the Fc fusion compositions comprising an immunogen such as an autoantigen or an allergen, are made recombinantly using nucleic acids encoding the compositions as is well known in the art. When the composition is a traditional therapeutic antibody, generally two nucleic acids, one encoding the heavy chain and one encoding the light chain, are introduced into a host cell (generally but not exclusively mammalian cells), and the host cells are grown under conditions whereby the antibodies are produced. Fusion compositions of the invention are generally made by constructing nucleic acids that encode the fusion (with or without protein linkers) as is well known in the art. In the case where the fusion compositions are chemically linked, separate nucleic acids encoding each domain may be made, introduced into host cells (either the same host cell or different ones) and grown under conditions where the proteins are expressed.

As will be appreciated by those in the art, the nucleic acids encoding the compositions of the invention may include other nucleic acid sequences including promoters and other regulatory sequences.

The compositions of the invention are then purified if required as is well known in the art.

Methods of Using the Compositions of the Invention

In a preferred embodiment, the compositions of the invention are administered to a patient to ameliorate, prevent or treat a disorder or disease. A “patient” for the purposes includes humans and other animals, preferably mammals and most preferably humans. By “disorder” or “disease” herein are meant a disorder that may be ameliorated by the administration of a pharmaceutical composition comprising a composition of the invention. When the composition of the invention is a therapeutic antibody containing a variant Fc domain, the disorder can be an antibody related disorder. Antibody related disorders include but are not limited to autoimmune diseases, immunological diseases, infectious diseases, inflammatory diseases, neurological diseases, and oncological and neoplastic diseases including cancer. By “cancer” and “cancerous” herein refer to or describe the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia and lymphoid malignancies.

In one embodiment, the compositions of the invention is the only therapeutically active agent administered to a patient. Alternatively, the compositions are administered in combination with one or more other therapeutic agents, including but not limited to cytotoxic agents, chemotherapeutic agents, cytokines, growth inhibitory agents, anti-hormonal agents, kinase inhibitors, anti-angiogenic agents, cardioprotectants, or other therapeutic agents. The compositions of the invention may be administered concomitantly with one or more other therapeutic regimens. For example, a composition of the invention useful in cancer (e.g. a therapeutic antibody) may be administered to the patient along with chemotherapy (including other therapeutic antibodies), radiation therapy, or both chemotherapy and radiation therapy. In addition, a variety of other therapeutic agents may find use for administration with the compositions of the invention, including any number of other drugs, including, but not limited to, antibodies, small molecule drugs and other biologics.

Pharmaceutical compositions are contemplated wherein a composition of the invention and one or more pharmaceutical carriers are formulated. Formulations of the IgG variants are prepared for storage by mixing the IgG having the desired degree of purity with optional pharmaceutically acceptable carriers, excipients or stabilizers (Remington's Pharmaceutical Sciences 16th edition, Osol, A. Ed., 1980, entirely incorporated by reference), in the form of lyophilized formulations or aqueous solutions. The formulations to be used for in vivo administration are preferably sterile. This is readily accomplished by filtration through sterile filtration membranes or other methods. The IgG variants and other therapeutically active agents disclosed herein may also be formulated as immunoliposomes, and/or entrapped in microcapsules.

The concentration of the composition in the formulation may vary from about 0.1 to 100% by weight. In a preferred embodiment, the concentration of the composition of the invention is in the range of 0.003 to 1.0 molar. In order to treat a patient, a therapeutically effective dose of the composition is administered. By “therapeutically effective dose” herein is meant a dose that produces the effects for which it is administered. The exact dose will depend on the purpose of the treatment, and will be ascertainable by one skilled in the art using known techniques. Dosages may range from 0.01 to 100 mg/kg of body weight or greater, for example 0.01, 0.1, 1.0, 10, or 50 mg/kg of body weight, with 1 to 10 mg/kg being preferred. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

Administration of the pharmaceutical compositions, preferably in the form of a sterile aqueous solution, may be done in a variety of ways, including, but not limited to, orally, subcutaneously, intravenously, parenterally, intranasally, intraotically, intraocularly, rectally, vaginally, transdermally, topically (e.g., gels, salves, lotions, creams, etc.), intraperitoneally, intramuscularly, intrapulmonary (e.g., AERx® inhalable technology commercially available from Aradigm, or Inhance® pulmonary delivery system commercially available from Nektar Therapeutics, etc.).

### EXAMPLES

Examples are provided below to illustrate the present invention. These examples are not meant to constrain the present invention to any particular application or theory of operation.

### Example 1

### Immunoprotection to Reduce Immunogencity of Biotherapeutics

This example describes the utilization of the FcγRIIb-mediated inhibitory mechanism to reduce the immune response to a therapeutic protein. Our novel approach, illustrated in FIG. 1, mimics the inhibitory effects of immune complex by high-affinity coengagement of FcγRIIb and the BCR coreceptor complex on human B cells. A key step in immune response to an adminstered protein is engagement with anti-drug specific BCR on B cells followed by activation, internalization, and presentation to T cells. Fusion of the therapeutic protein to the IgG Fc region enables interaction with the inhibitory receptor FcγRIIb. However, because native IgG's bind FcγRIIb with weak (uM) affinity, FcγRIIb-mediated inhibition occurs in response only to immune complexed but not monomeric IgG Fc.

Central to our strategy is high affinity interaction between the Fc region and FcγRIIb, which may enable maximal inhibition of B cell activation by monovalent (non-immune complexed) therapeutic protein). Coupling of FcγRIIb-enhanced (IIbE) Fc domains to therapeutic proteins utilizes the natural inhibitory pathway to inhibit the B cell response to the fusion partner. Enhanced affinity of the protein-Fc fusion for the inhibitory FcγRIIb will prevent anti-therapeutic B cells from activation and differentiation into immunoglobulin-producing plasma cells.

The Fc domain was engineered to create a biologic with high affinity for FcγRIIb that mimics the suppressive effects of cognate immune complex on activated B cells (U.S. Ser. No. 12/156,183, filed May 30, 2008, entitled “Methods and Compositions for Inhibiting CD32b Expressing cells”, herein incorporated expressly by reference). B cells are important in adaptive immunity because BCR-antigen complex internalization is the first step in antigen presentation. Because FcγRIIb coengagement inhibits BCR-dependent antigen internalization and processing, the activities of the IIbE variants presented here may also suppress T cell-mediated adaptive immunity. The fusions of the invention thus suppress differentiation, survival, and proliferation only of B cell populations possessing BCRs specific for epitopes of the original (non-modified) drug. This strategy may selectively eliminate only drug-reactive B cells, and thus mitigate potential immunogenicity concerns arising from the clinical use of therapeutic proteins. Thus IIbE Fc regions have therapeutic applications by selectively eliminating only drug-reactive B cells, and thus mitigate potential immunogenicity concerns arising from the clinical use of therapeutic proteins.

The exemplary test system for the immunoprotection approach outlined herein was the anti-TNFα (anti-TNF) antibody adalimumab (marketed as the drug Humira®). Despite being engineered as a fully human amino acid sequence, Humira is known to elicit significant immunogenicity in humans. Approximately 5.5% of patients with rheumatoid arthritis under combination treatment with adalimumab and methotrexate (MTX) developed an unwanted immune response, and without concomitant methotrexate the incidence of an unwanted immune response was 12.4% (European Medicines Agency, 2008, European Public Assessment Report (EPAR) for Humira®). Immunogenicity of adalimumab negatively affects its clinical outcome (West et al., 2008, Alimentary Pharmacology & Therapeutics 28:1122-1126.

Genes encoding the heavy and light VH and VL domains of adalimumab were synthesized commercially (Blue Heron Biotechnologies) and subcloned into the mammalian expression vector pTT5 (National Research Council Canada) encoding the IgG1 heavy chain constant region and Cκ constant region respectively. Heavy and light chain constructs were cotransfected into HEK293E cells for expression, and antibodies were purified using protein A affinity chromatography (Pierce Biotechnology, Rockford, Ill.).

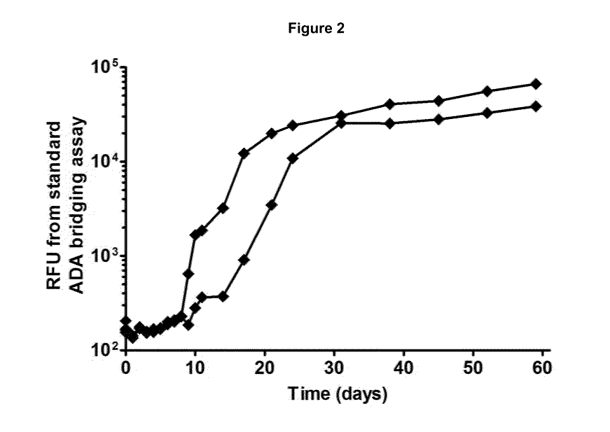

Adalimumab IgG1 antibody was tested for anti-drug antibody (ADA) response in non-human primates, an experimental surrogate for clinical studies in humans. In-life portions were conducted at SNBL USA, LTD. Two male cynomolgus monkeys (macaca fascicularis) weighing 3.5-4.5 kg were given two 20 mg/kg intravenous doses of rituximab IgG1 on days −21 and −7 in order to deplete B cells, followed by a single 4 mg/kg intravenous dose of adalimumab IgG1 on day 1. Blood samples (1 ml) were drawn from 5 minutes to 60 days after completion of the infusion, processed to serum and stored at −70° C. Immunoassays to detect ADA were carried out at Xencor using a bridging assay. Plates were coated with adalimumab IgG1 antibody, and then blocked with SuperBlock (Pierce) and washed with buffer. Cyno serum samples were added in 5-fold dilutions, as well as recombinant TNFα (R&D Systems) as a controls, and plates were incubated at room temperature for 1 hour. Plates were washed, and anti-adalimumab antibody was detected by adding europium-labeled adaliumumab. Plates were incubated at room temperature for 1 hour, washed, DELFIA Enhancement Solution (Perkin Elmer) was added, and samples were incubated at room temperature for 15 minutes in the dark. Time-resolved flluorescence was read using an Envision plate reader (Perkin Elmer). The results in FIG. 2 show that adalimumab IgG1 elicited strong immune response in both monkeys despite B cell depletion using rituximab.

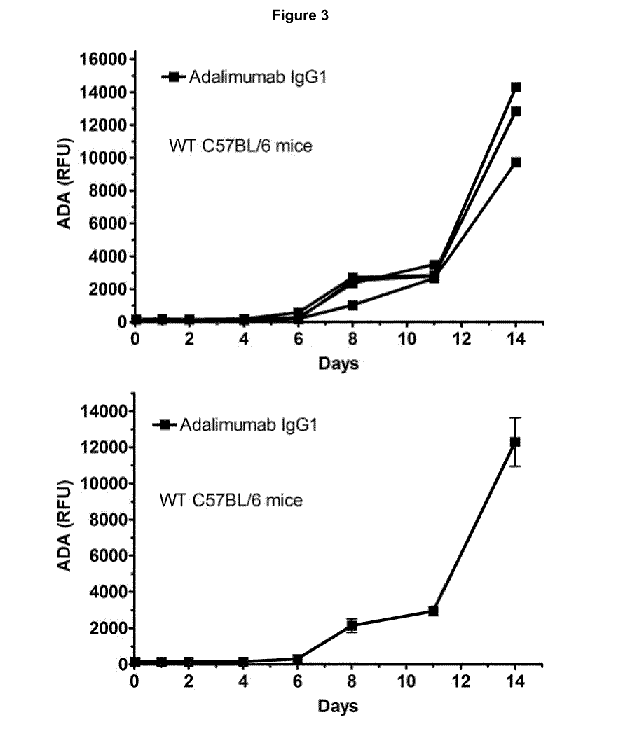

A similar study was carried out in C57BL/6 mice. Four mice were given a single intravenous 2 mg/kg dose, and 25-50 ul blood samples were collected via retro-orbital sinus/plexus (OSP) at times 1 hr and days 1, 2, 4, 6, 8, 11, and 14 post-injection. Anti-adalimumab antibody was detected using the ADA assay described above. Results in FIG. 3 show, 100% immunogenicity of IgG1 adalimumab in mice, similar to the results of the cyno data. Together the results of the cyno and mouse experiments indicated that adalimumab was a good test system for the immunoprotection approach.

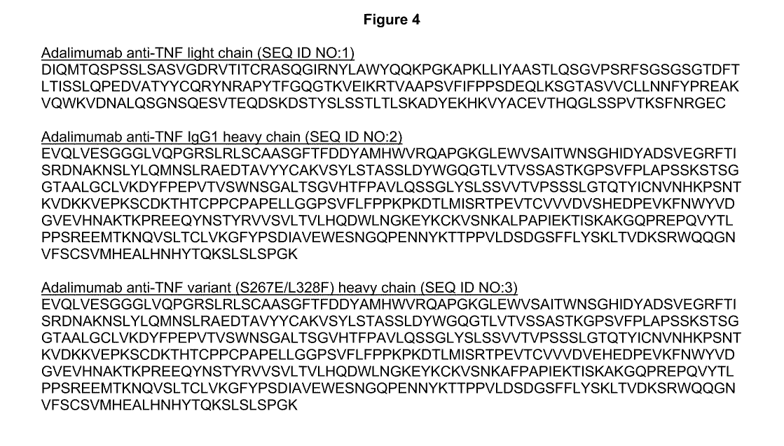

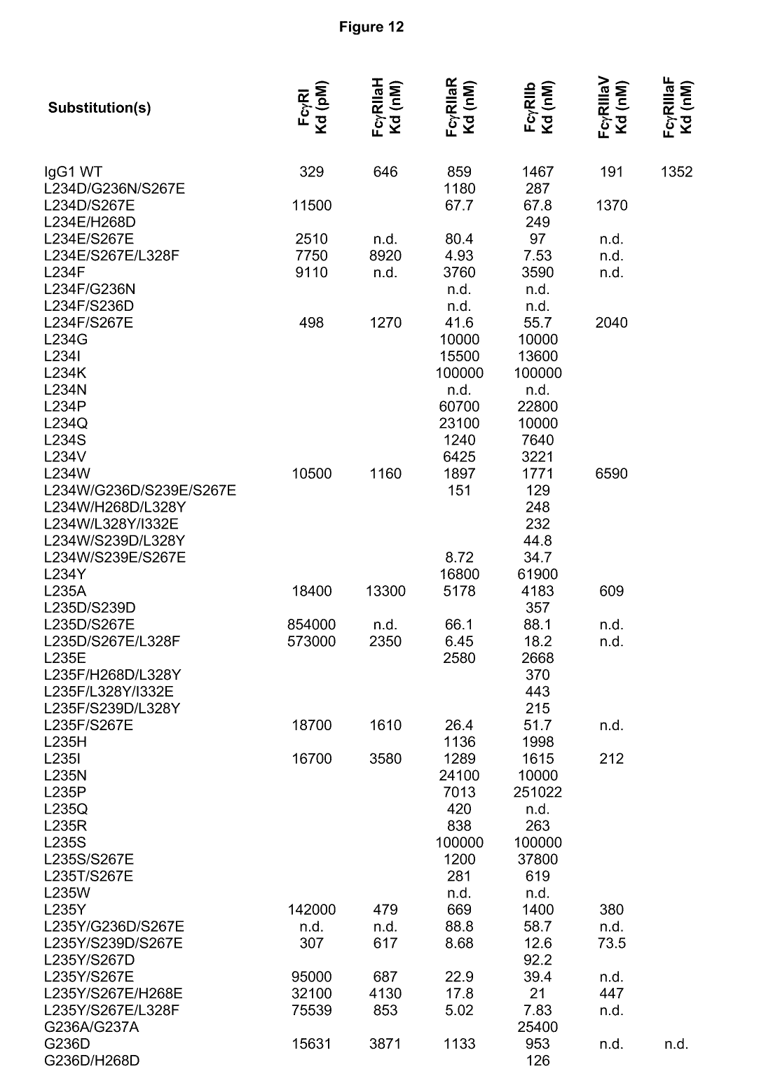

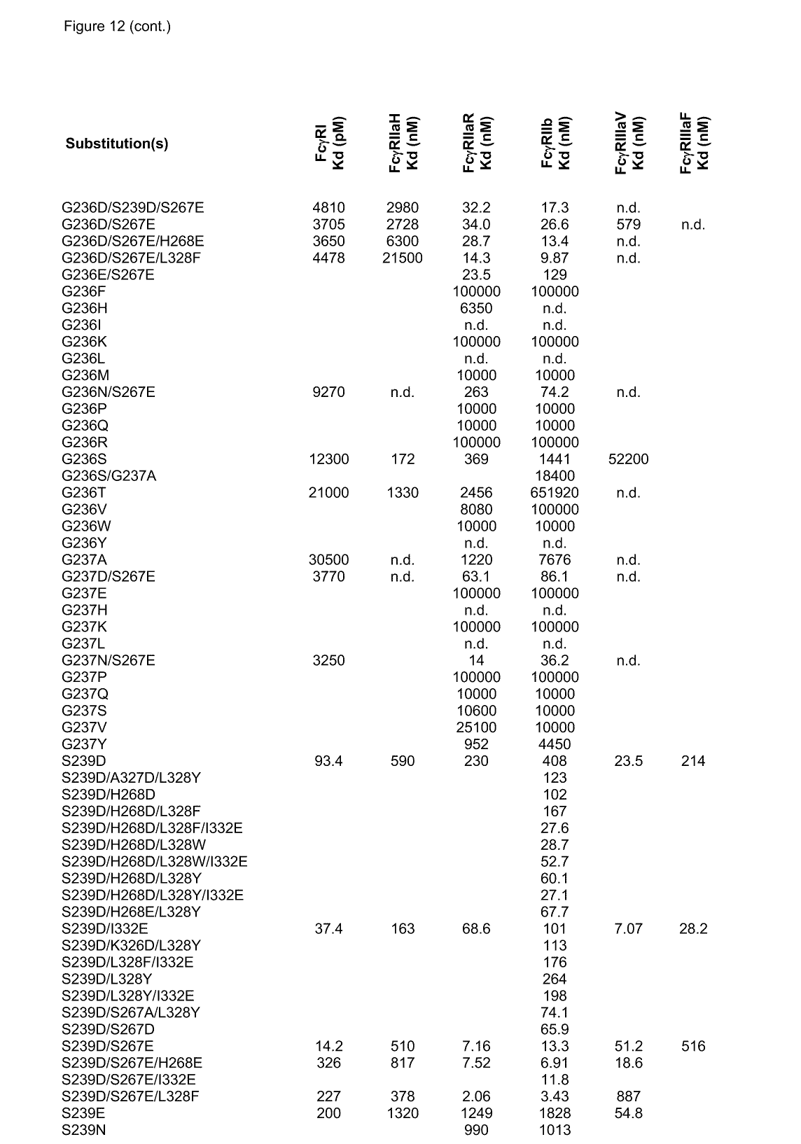

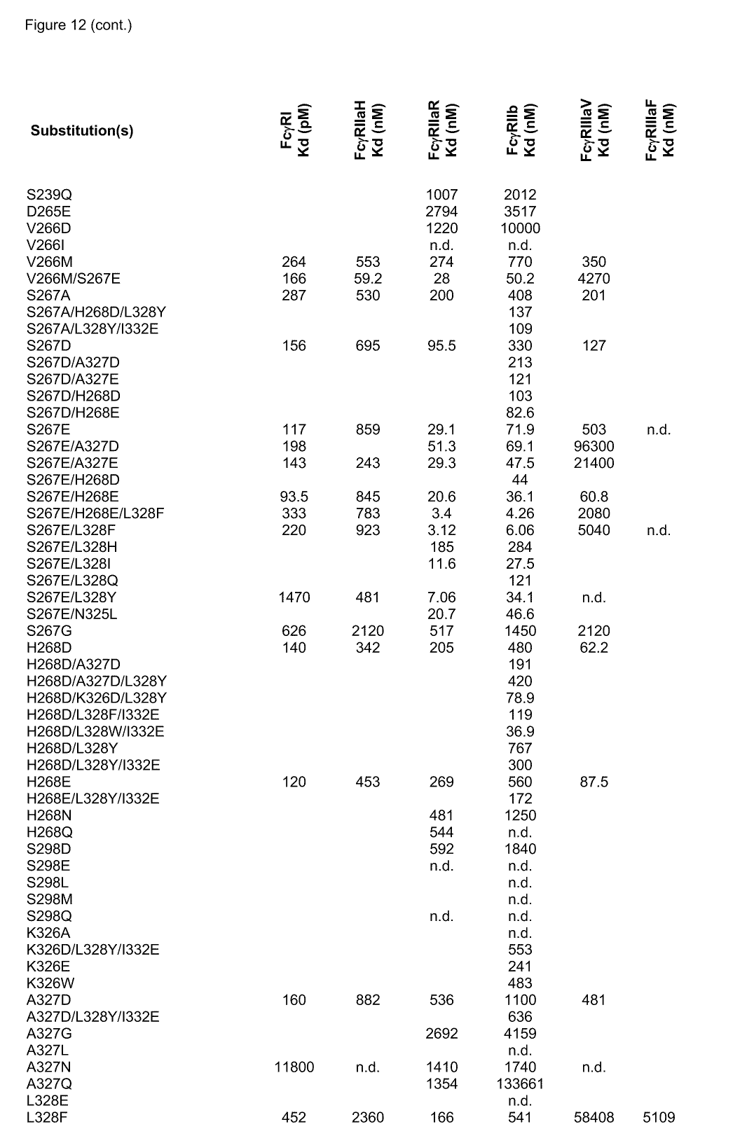

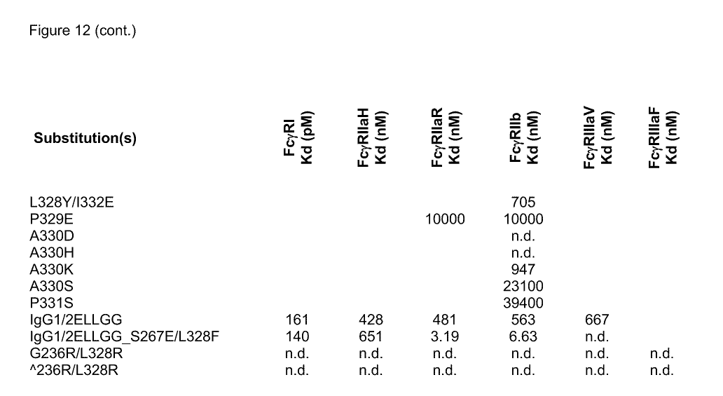

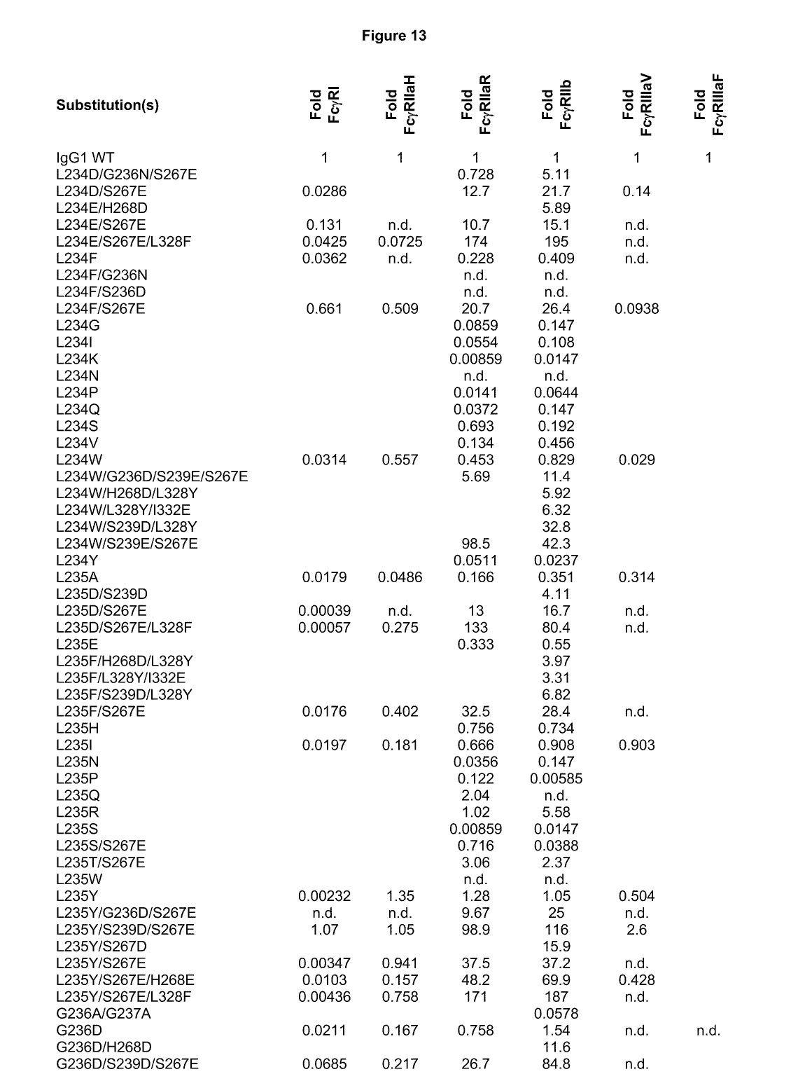

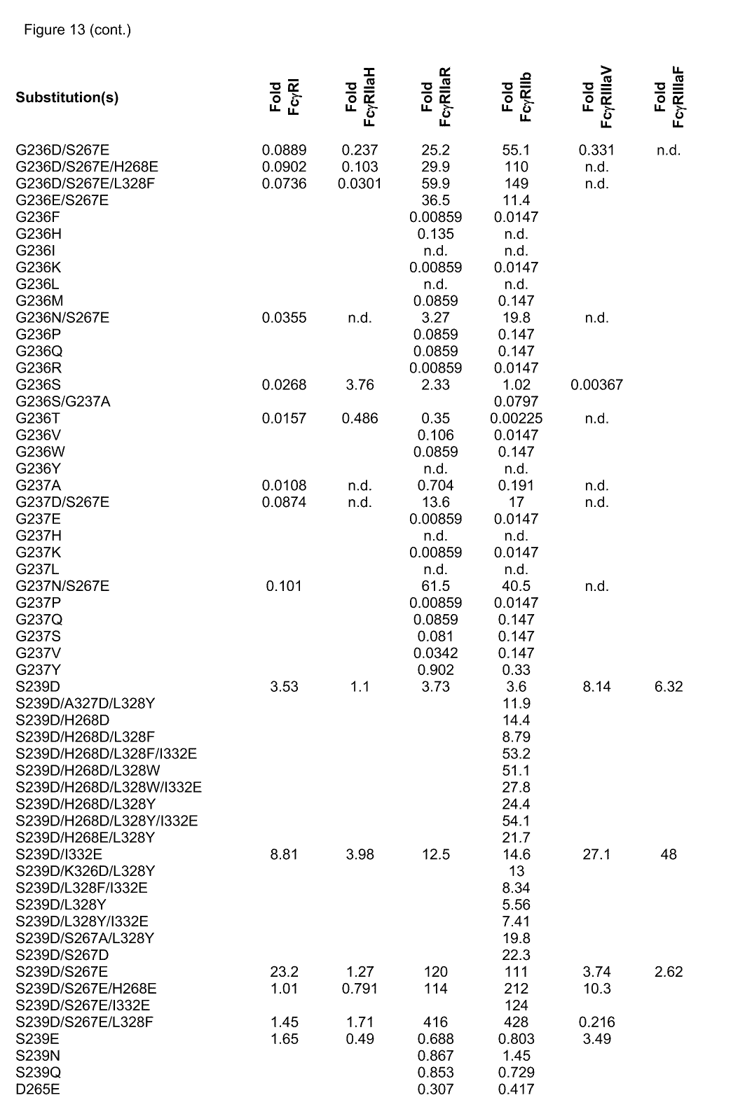

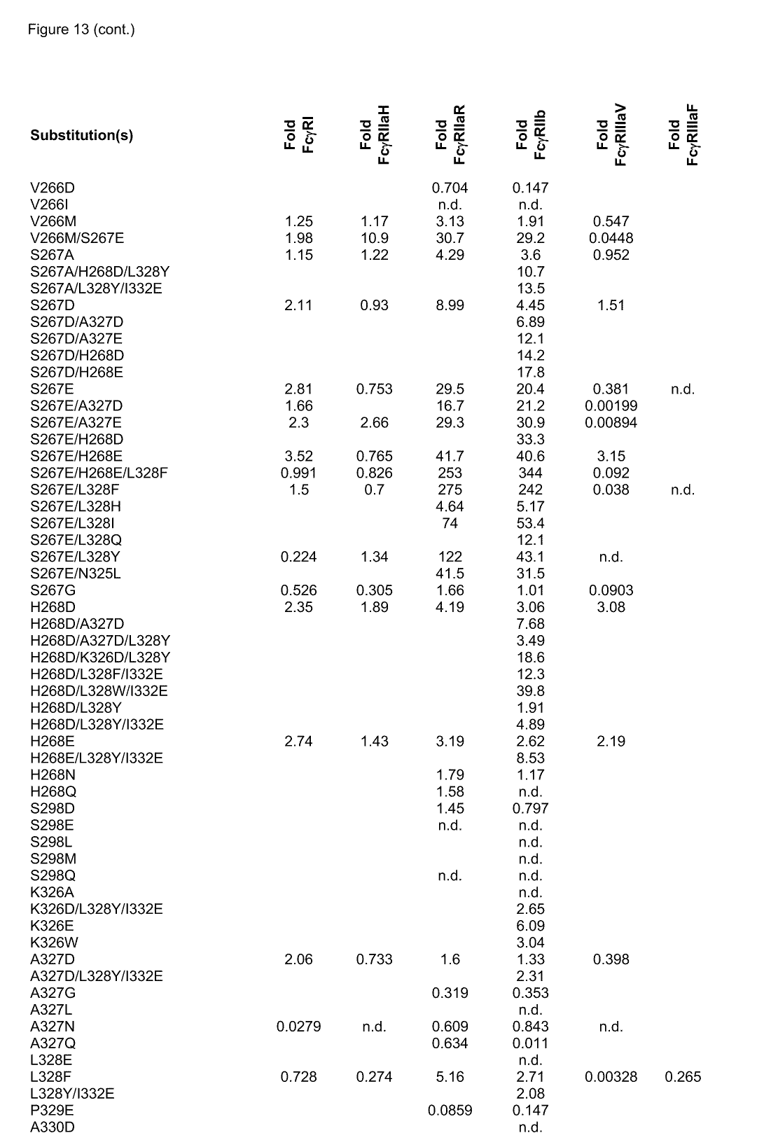

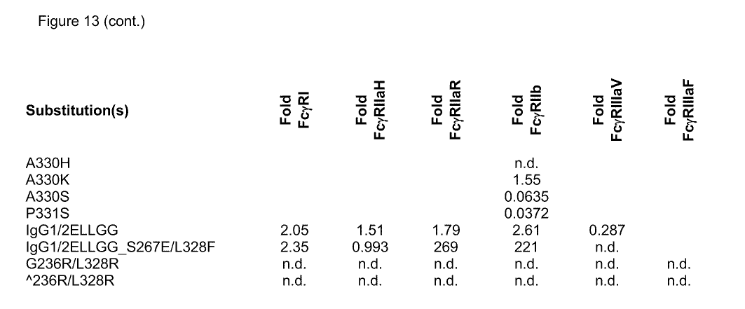

Under physiological conditions, bridging of the BCR with FcγRIIb and subsequent B cell suppression occurs via immune complexes of IgGs and cognate antigen. The design strategy was to reproduce this effect using a single crosslinking antibody. Human IgG binds human FcγRIIb with weak affinity (greater than 100 nM for IgG1), and FcγRIIb-mediated inhibition occurs in response to immune-complexed but not monomeric IgG. We reasoned that high affinity to this receptor (less than 100 nM) would be required for maximal inhibition of B cell activation. Engineered Fc variants have been described that bind to FcγRIIb with improved affinity relative to native IgG1 (U.S. Ser. No. 12/156,183, filed May 30, 2008, entitled “Methods and Compositions for Inhibiting CD32b Expressing cells”, herein incorporated expressly by reference). In order to enhance the inhibitory activity of adalimumab, the Fc region was engineered with Fc variant 267E/328F that improves binding to FcγRIIb. FIG. 4 provides amino acid sequences of the light and heavy chains of IgG1 and Fc-engineered adalimumab. As outlined herein, other variants also find use in this strategy; Table 1 shows the K_{D}s for some of the other variants outlined herein, although as will be appreciated by those in the art, any number of variants (many of which are included in FIGS. 12 and 13) can be used.

TABLE 1
FcγRIIb binding affinities of Fc variants

| | FcγRIIb | | FcγRIIb | |
|---|---|---|---|---|
| | Biacore | | Cell Surface | |
| | K_{D} (μM)^{†} | Fold^{‡} | EC₅₀ (μM) | Fold^{‡} |
| Native IgG1 | 1.8 ± 0.5 | 1 | 0.44 | 1 |
| G236D | 1.0 ± 0.3 | 1.8 | n.d.^{¶} | — |
| L328F | 0.64 ± 0.18 | 2.8 | n.d.^{¶} | — |
| S239D | 0.38 ± 0.06 | 4.7 | n.d.^{¶} | — |
| S267E | 0.060 ± 0.005 | 30 | 0.025 | 18 |
| G236D/S267E | 0.025 ± 0.001 | 72 | 0.0060 | 74 |
| S239D/S267E | 0.012 ± 0.001 | 150 | 0.0020 | 220 |
| S267E/L328F | 0.0042 ± 0.0004 | 430 | 0.0014 | 320 |

^{†}K_{D}s were from global Langmuir fits of SPR data (mean ± SD). SDs from n = 4 for FcγRIIb, n = 2 for other Fcγ receptors.
^{‡}Fold = K_{D} (Native IgG1)/K_{D} (variant).
^{¶}Not determined.

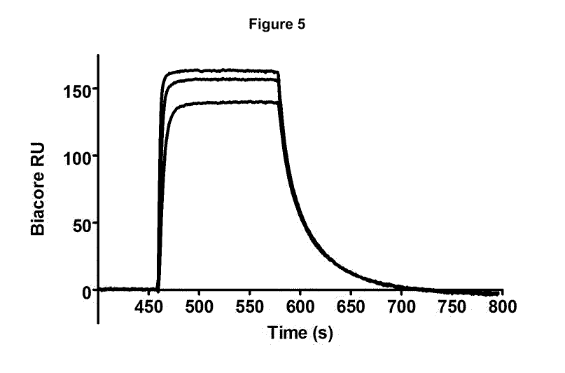

S267E and L328F substitutions were introduced into adalimumab IgG1 in the pTT5 vector using site-directed mutagenesis (QuikChange, Stratagene, Cedar Creek, Tex.). Heavy and light chain constructs were cotransfected into HEK293E cells for expression, and antibodies were purified using protein A affinity chromatography (Pierce Biotechnology, Rockford, Ill.). Binding to human FcγRIIb and TNFα antigen were measured using surface plasmon resonance (SPR) based technology (Biacore). SPR measurements were performed using a Biacore 3000 instrument (Biacore, Piscataway, N.J.). A protein NG (Pierce Biotechnology) CM5 biosensor chip (Biacore) was generated using a standard primary amine coupling protocol. All measurements were performed using HBS-EP buffer (10 mM HEPES pH 7.4, 0.15 M NaCl, 3 mM EDTA, 0.005% vol/vol surfactant P20, Biacore). Antibodies at 20 nM or 50 nM in HBS-EP buffer were immobilized on the protein A/G surface, and then recombinant human FcγRIIb (R&D Systems) or TNFα (R&D Systems) was injected in a concentration series. After each cycle, the surface was regenerated by injecting glycine buffer (10 mM, pH 1.5). Data were processed by zeroing time and response before the injection of analyte and by subtracting appropriate nonspecific signals (response of reference channel and injection of running buffer). Kinetic analyses were performed by global fitting of binding data with a 1:1 Langmuir binding model using BIAevaluation software (Biacore). Sensorgrams for binding to FcγRIIb are shown in FIG. 5, and fitted affinities for both TNFα and FcγRIIb are provided in Table 2.

TABLE 2
Affinities of Fc-engineered adalimumab
for human TNF antigen and human FcγRIIb

| | TNF | FcγRIIb |
|---|---|---|
| | KD (M) | KD (M) |
| Adalimumab_IgG1_S267E/L328F | 1.5 × 10−09 | 1.4 × 10−09 |

Whereas WT IgG1 Fc binds with FcγRIIb with μM affinity (KD=1.8 uM, Chu et al., 2008, Molecular Immunology 45:3926-3933), the S267E/L328F variant enhances binding to FcγRIIb over two orders of magnitude to nM affinity. Consistent with previous results, the Fc engineered adalimumab bound FcγRIIb with an affinity of 1.4 nM. Binding to TNFα antigen was unchanged from IgG1.

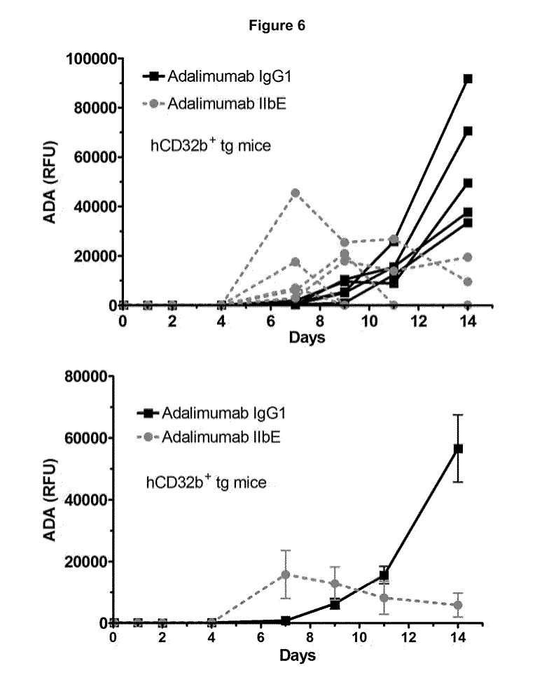

Immunogenicity of IgG1 and FcγRIIb-enhanced (267E/328F, also referred to as “IIbE” herein) versions of adalimumab were tested in vivo in mice. Because the 267E/328F variant does not substantially enhance affinity of the Fc region to murine FcγRIIb, the study was carried out in human FcγRIIb transgenic B6.129S FcgR2btm1Rav mice (from Dr. Jeffrey V. Ravetch, Molecular Genetics and Immunology, Rockefeller University, New York). These mice are missing mouse FcγRIIb but have human FcγRIIb, and are referred to as hFcγRIIb+tg mice. Five mice were given a single intravenous 2 mg/kg dose, and 25-50 ul blood samples were collected via retro-orbital sinus/plexus (OSP) at times 1 hr and days 1, 2, 4, 7, 9, 11, and 14 post-injection. Anti-adalimumab antibody was detected using the ADA assay described above. Results are shown in FIG. 6. Consistent with the previous studies, the IgG1 version of adalimumab showed 100% immune response. In contrast, the IIbE version of adalimumab engineered for high affinity to FcγRIIb showed a substantial reduction in immunogenicity. Mean ADA response on day 14 was 56640 for IgG1 and 5877 for IIbE. Thus the IIbE Fc region resulted in a 90% (10-fold) reduction in ADA response.

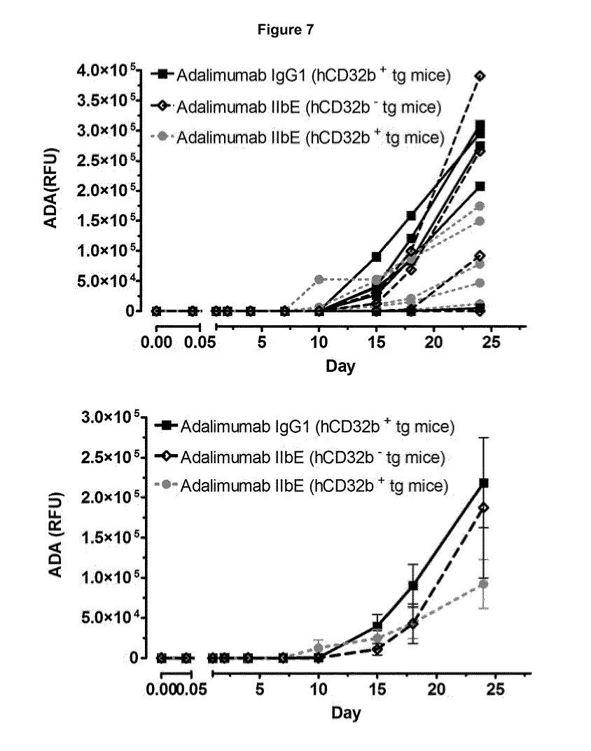

In order to further test the contribution of enhanced FcγRIIb affinity to the observed immunoprotection effect of the Fc-engineered adalimumab biotherapeutic, the immunogenicity study hCD32b tg+ mice was repeated using littermate hCD32b tg− mice, which are a genetically identical match to the hCD32b tg+ mice used in the previous study except that they lack the human CD32b gene. A higher 10 mg/kg dose was used for this study. Five mice were given a single intravenous 10 mg/kg dose, and 25-50 ul blood samples were collected via retro-orbital sinus/plexus (OSP) at times 1 hr and days 1, 2, 5, 8, 12, 15, and 18 post-injection. Anti-adalimumab antibody was detected using the ADA assay described above. The results are shown in FIG. 7. As previously observed, adalimumab IgG1 resulted in a strong immune response in the hCD32b+tg mice. Again, Fc-engineered adalimumumab IIbE (267E/328F) showed a reduction in the level of immunogenicity in the hCD32b+tg mice. Mean ADA response on day 24 was 218478 for IgG1 and 92092 for IIbE. Thus the IIbE Fc region resulted in a 58% (2.4-fold) reduction in ADA response. In mice lacking the human FcγRIIb transgene (hCD32b−tg mice), however, adalimumab IIbE did not reduce immunogenicity. These results firmly support the role of high affinity FcγRIIb binding in the immunoprotective effect of the variant biotherapeutic.

A summary of the results of the two experiments in hCD32b+tg mice is provided in Table 3. For the first in vivo experiment (2 mg/kg dose), mean ADA response on the final day of the study (day 14) was 56640 for IgG1 and 5877 for IIbE. The cutpoint of the ADA assay was approximately 200 RFU (relative fluorescence units). The mean RFU's for the five mice for the IgG1 and IIbE groups on the final day of the study (day 14) were thus 283-fold and 29-fold relative to cutpoint respectively. Thus reduction in ADA between IgG1 (RFU=56640) and IIbE (RFU=5877) was 90%, and thus the IIbE Fc region resulted in a 10-fold reduction in ADA response. For the second in vivo experiment (10 mg/kg dose), mean ADA response on the final day of the study (day 24) for the hCD32b+tg mice was 218478 for IgG1 and 92092 for IIbE. The cutpoint of the ADA assay was approximately 200 RFU (relative fluorescence units). The mean RFU's for the five mice for the IgG1 and IIbE groups on the final day were thus 1092-fold and 460-fold relative to cutpoint respectively. Thus reduction in ADA between IgG1 (RFU=218478) and IIbE (RFU=92092) was 58%, and thus the IIbE Fc region resulted in a 2.4-fold reduction in ADA response.

TABLE 3
Results of ADA experiments in hCD32b⁺ tg mice

| | | | | Final | Final | Fold | % | Fold |
|---|---|---|---|---|---|---|---|---|
| Dose | | Cutpoint | Final | Mean | Mean | Final/ | ADA | ADA |
| mg/kg | mAb | (RFU) | Day | RFU | SD | Cutpoint | Reduction | Reduction |
| 2 | Adalimumab | 200 | 14 | 56640 | 24380 | 283 | | |
| | IgG1 | | | | | | | |
| 2 | Adalimumab | 200 | 14 | 5877 | 8635 | 29 | 89.6% | 9.6 |
| | IIbE | | | | | | | |
| 10 | Adalimumab | 200 | 24 | 218478 | 125513 | 1092 | | |
| | IgG1 | | | | | | | |
| 10 | Adalimumab | 200 | 24 | 92092 | 68416 | 460 | 57.8% | 2.4 |
| | IIbE | | | | | | | |

In addition, the variants herein, and in particular 267E/328F, can be transferred to other therapeutic antibodies and exhibit the same effects. Previous work has shown that once identified, Fc variants can be “transferred” to different antibodies, including antibodies with different Fv regions and different isotypes and subclasses of Igs, and retain their function. See for example U.S. Ser. No. 12/156,183, filed May 30, 2008, entitled “Methods and Compositions for Inhibiting CD32b Expressing cells”, and U.S. Ser. No. 12/562,088, filed Sep. 17, 2009, entitled “Novel Compositions and Methods for Treating IgE-Mediated Disorders”, herein incorporated expressly by reference.

### Example 2

### Immunoprotection to Reduce Immune Response to an Autoimmune Antigen or Allergen

We chose as our test system for immunoprotection of an autoantigen myelin oligodendrocyte glycoprotein (MOG). MOG is a glycoprotein of the myelin sheath that has been intensively studied as an autoantigen in demyelinating diseases such as multiple sclerosis (MS) (Lalive, 2008, Swiss Med Wkly 138[47-48]:692-707). The amino acid sequence of the extracellular domain (ECD) of human MOG is provided in FIG. 10. We designed genetic constructs in which the gene encoding the MOG ECD was fused to the hinge and Fc region of IgG1, as well as the variant Fc region comprising the 267E and L328F that provide enhanced affinity for human FcγRIIb (FIG. 10). Recent advances have shown that epitope specificity of MOG is crucial in terms of specificity of the antibody response (Lalive, 2008, Swiss Med Wkly 138[47-48]:692-707). Thus rather than full length MOG-Fc fusions, fusions of specific MOG peptides to Fc may also be used for the immunoprotection approach. We also designed Fc fusions to MOG(35-55) peptide. In this case murine MOG(35-55) was used as it is useful for pre-clinical testing of the immunoprotection approach. Fc fusions may of course be generated with human MOG peptides.

Genes encoding the human MOG protein and mouse MOG(35-55) peptide were synthesized commercially (Blue Heron Biotechnologies) and subcloned into the mammalian expression vector pTT5 (National Research Council Canada) encoding the IgG1 hinge and Fc region, as well as the Fc-engineered 267E/328F IgG1 Fc region. In addition, as a negative control the MOG and MOG peptide genes were fused to Fc regions containing two substitutions 236R and 328R that ablate binding to all Fc receptors, referred to as Fc(KO). Genes were transfected into HEK293E cells for expression, and Fc fusions were purified using protein A affinity chromatography (Pierce Biotechnology, Rockford, Ill.).

Binding to the human inhibitory receptor FcγRIIb, the human activating receptor FcγRIIIa, and an antibody recognizing human MOG were measured using surface plasmon resonance. SPR measurements were performed using a Biacore 3000 instrument (Biacore, Piscataway, N.J.). A protein A/G (Pierce Biotechnology) CM5 biosensor chip (Biacore) was generated using a standard primary amine coupling protocol. All fusion proteins were diluted in HBS-EP buffer to 100 nM and immobilized on protein A followed by injection of FcγRIIb, FcγRIIIa, or anti-MOG antibody. Anti-MOG antibody is a rat IgG2b which does not bind to protein A, and thus does not interference with the binding experiment. Data were processed by zeroing time and response before the injection of analyte and by subtracting appropriate nonspecific signals (response of reference channel and injection of running buffer).

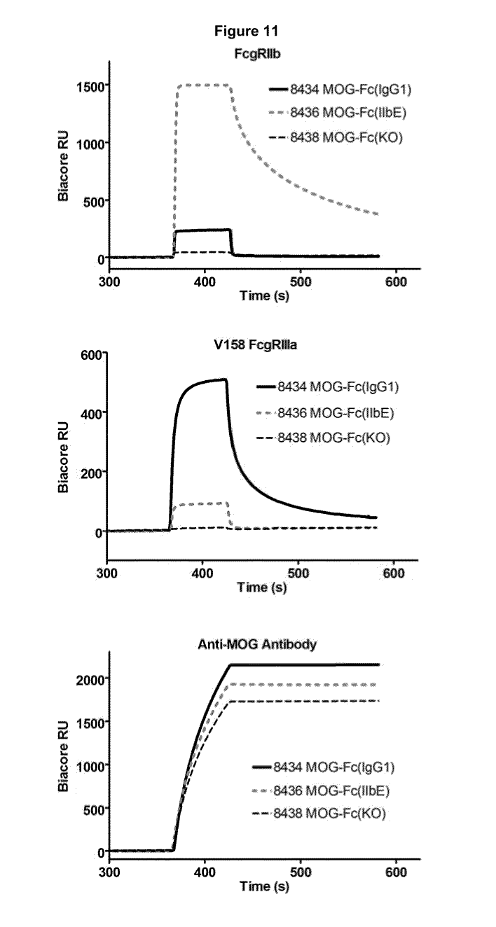

Sensorgrams for binding to FcγRIIb are shown in FIG. 11. The native IgG1 Fc fusion binds weakly to human FcγRIIb, and the Fc(KO) version does not bind at all, consistent with previous results. In contrast, the IIbE variant (267E/328F) provides high affinity binding to the MOG-Fc fusion. Native IgG1 Fc fusion binds with moderate affinity to the V158 isoform of the activating human Fc receptor FcγRIIIa, while the IIbE variant has substantially reduced binding, consistent with previous results. The Fc(KO) version does not bind FcγRIIIa. All three Fc fusion constructs bind anti-MOG antibody equivalently, indicating that fusion to Fc does not impact the fidelity of MOG protein conformation.

### [1] REFERENCES

1. DeFranco A L (1997) The complexity of signaling pathways activated by the BCR. Curr Opin Immunol 9:296-308.
2. Pierce S K (2002) Lipid rafts and B-cell activation. Nat Rev Immunol 2:96-105.
3. Ravetch J V, Lanier L L (2000) Immune inhibitory receptors. Science 290:84-89.
4. Doody G M, Dempsey P W, Fearon D T (1996) Activation of B lymphocytes: integrating signals from CD19, CD22 and FcgRIIb1. Curr Opin Immunol 8:378-382.
5. L1 D H, et al. (2006) CD72 down-modulates BCR-induced signal transduction and diminishes survival in primary mature B lymphocytes. J Immunol 176:5321-5328.
6. Heyman B (2003) Feedback regulation by IgG antibodies. Immunol Lett 88:157-161.
7. Chan P L, Sinclair N R (1973) Regulation of the immune response. VI. Inability of F(ab′)2 antibody to terminate established immune responses and its ability to interfere with IgG antibody-mediated immunosuppression. Immunology 24:289-301.
8. Amigorena S, et al. (1992) Cytoplasmic domain heterogeneity and functions of IgG Fc receptors in B lymphocytes. Science 256:1808-1812.
9. Muta T, et al. (1994) A 13-amino-acid motif in the cytoplasmic domain of FcgRIIB modulates B-cell receptor signalling. Nature 368:70-73.
10. Kiener P A, et al. (1997) Co-ligation of the antigen and Fc receptors gives rise to the selective modulation of intracellular signaling in B cells. Regulation of the association of phosphatidylinositol 3-kinase and inositol 5′-phosphatase with the antigen receptor complex. J Biol Chem 272:3838-3844.
11. Ono M, Bolland S, Tempst P, Ravetch J V (1996) Role of the inositol phosphatase SHIP in negative regulation of the immune system by the receptor FcgRIIB. Nature 383:263-266.
12. Tridandapani S, Phee H, Shivakumar L, Kelley T W, Coggeshall K M (1998) Role of SHIP in FcgRIIb-mediated inhibition of Ras activation in B cells. Mol Immunol 35:1135-1146.
13. Wernersson S, et al. (1999) IgG-mediated enhancement of antibody responses is low in Fc receptor g chain-deficient mice and increased in FcgRII-deficient mice. J Immunol 163:618-622.
14. Yuasa T, et al. (1999) Deletion of Fcg receptor IIB renders H-2b mice susceptible to collagen-induced arthritis. J Exp Med 189:187-194.
15. Fukuyama H, Nimmerjahn F, Ravetch J V (2005) The inhibitory Fcg receptor modulates autoimmunity by limiting the accumulation of immunoglobulin G+anti-DNA plasma cells. Nat Immunol 6:99-106.
16. McGaha T L, Sorrentino B, Ravetch J V (2005) Restoration of tolerance in lupus by targeted inhibitory receptor expression. Science 307:590-593.
17. Nakamura A, et al. (2000) Fcg receptor IIB-deficient mice develop Goodpasture's syndrome upon immunization with type IV collagen: A novel murine model for autoimmune glomerular basement membrane disease. J Exp Med 191:899-906.
18. Blank M C, et al. (2005) Decreased transcription of the human FCGR2B gene mediated by the −343 G/C promoter polymorphism and association with systemic lupus erythematosus. Hum Genet. 117:220-227.
19. Olferiev M, Masuda E, Tanaka S, Blank M C, Pricop L (2007) The role of activating protein 1 in the transcriptional regulation of the human FCGR2B promoter mediated by the −343 G->C polymorphism associated with systemic lupus erythematosus. J Biol Chem 282:1738-1746.
20. Chen J Y, et al. (2006) Association of a transmembrane polymorphism of Fcg receptor IIb (FCGR2B) with systemic lupus erythematosus in Taiwanese patients. Arthritis Rheum 54:3908-3917.
21. Floto R A, et al. (2005) Loss of function of a lupus-associated FcgRIIb polymorphism through exclusion from lipid rafts. Nat Med 11:1056-1058.
22. L1 X, et al. (2003) A novel polymorphism in the Fcg receptor IIB (CD32B) transmembrane region alters receptor signaling. Arthritis Rheum 48:3242-3252.
23. Mackay M, et al. (2006) Selective dysregulation of the FcgIIB receptor on memory B cells in SLE. J Exp Med 203:2157-2164.
24. Su K, et al. (2007) Expression profile of FcgRIIb on leukocytes and its dysregulation in systemic lupus erythematosus. J Immunol 178:3272-3280.
25. Pritchard N R, Smith K G (2003) B cell inhibitory receptors and autoimmunity. Immunology 108:263-273.
26. Stefanescu R N, Olferiev M, Liu Y, Pricop L (2004) Inhibitory Fcg receptors: From gene to disease. J Clin Immunol 24:315-326.
27. Lazar G A, et al. (2006) Engineered antibody Fc variants with enhanced effector function. Proc Natl Acad Sci USA 103:4005-4010.
28. Fujimoto M, Poe J C, Hasegawa M, Tedder T F (2001) CD19 amplification of B lymphocyte Ca2+ responses: a role for Lyn sequestration in extinguishing negative regulation. J Biol Chem 276:44820-44827.
29. Fulcher D A, Basten A (1994) Reduced life span of anergic self-reactive B cells in a double-transgenic model. J Exp Med 179:125-134.
30. Desjarlais J R, Lazar G A, Zhukovsky E A, Chu S Y (2007) Optimizing engagement of the immune system by anti-tumor antibodies: an engineers perspective. Drug Discov Today 12:898-910.
31. Kim S J, Park Y, Hong H J (2005) Antibody engineering for the development of therapeutic antibodies. Mol Cells 20:17-29.
32. Carter N A, Harnett M M (2004) Dissection of the signalling mechanisms underlying FcgRIIB-mediated apoptosis of mature B-cells. Biochem Soc Trans 32:973-975.
33. Van Den Herik-Oudijk I E, Westerdaal N A, Henriquez N V, Capel P J, Van De Winkel J G (1994) Functional analysis of human FcgRII (CD32) isoforms expressed in B lymphocytes. J Immunol 152:574-585.
34. Ferrara C, Stuart F, Sondermann P, Brunker P, Umana P (2006) The carbohydrate at FcgRIIIa Asn-162. An element required for high affinity binding to non-fucosylated IgG glycoforms. J Biol Chem 281:5032-5036.
35. Masuda K, et al. (2007) Enhanced binding affinity for FcgRIIIa of fucose-negative antibody is sufficient to induce maximal antibody-dependent cellular cytotoxicity. Mol. Immunol.
36. Kaneko Y, Nimmerjahn F, Ravetch J V (2006) Anti-inflammatory activity of immunoglobulin G resulting from Fc sialylation. Science 313:670-673.
37. Tarasenko T, Dean J A, Bolland S (2007) FcgRIIB as a modulator of autoimmune disease susceptibility. Autoimmunity 40:409-417.
38. Samuelsson A, Towers T L, Ravetch J V (2001) Anti-inflammatory activity of IVIG mediated through the inhibitory Fc receptor. Science 291:484-486.
39. Edwards J C, et al. (2004) Efficacy of B-cell-targeted therapy with rituximab in patients with rheumatoid arthritis. N Engl J Med 350:2572-2581.
40. Hauser S L, et al. (2008) B-cell depletion with rituximab in relapsing-remitting multiple sclerosis. N Engl J Med 358:676-688.
41. Bouaziz J D, et al. (2007) Therapeutic B cell depletion impairs adaptive and autoreactive CD4+T cell activation in mice. Proc Natl Acad Sci USA 104:20878-20883.
42. Lanzavecchia A (1996) Mechanisms of antigen uptake for presentation. Curr Opin Immunol 8:348-354.
43. Watts C (1997) Capture and processing of exogenous antigens for presentation on MHC molecules. Annu Rev Immunol 15:821-850.
44. Minskoff S A, Matter K, Mellman 1 (1998) FcgRII-B1 regulates the presentation of B cell receptor-bound antigens. J Immunol 161:2079-2083.
45. Wagle N M, Faassen A E, Kim J H, Pierce S K (1999) Regulation of B cell receptor-mediated MHC class II antigen processing by FcgRIIB1. J Immunol 162:2732-2740.
46. Mertsching E, et al. (2008) A mouse Fcg-Fcc protein that inhibits mast cells through activation of FcgRIIB, SH2 domain-containing inositol phosphatase 1, and SH2 domain-containing protein tyrosine phosphatases. J Allergy Clin Immunol 121:441-447.
47. Allen L C, Kepley C L, Saxon A, Zhang K (2007) Modifications to an Fcg-Fce fusion protein alter its effectiveness in the inhibition of FceRI-mediated functions. J Allergy Clin Immunol 120:462-468.
48. Meeker T C, et al. (1984) A unique human B lymphocyte antigen defined by a monoclonal antibody. Hybridoma 3:305-320.
49. Lazar G A, Desjarlais J R, Jacinto J, Karki S, Hammond P W (2007) A molecular immunology approach to antibody humanization and functional optimization. Mol Immunol 44:1986-1998.
50. Kabat E A, Wu T T, Perry H M, Gottesman K S, Foeller C (1991) Sequences of Proteins of Immunological Interest (U.S. Department of Health and Human Services, Bethesda).
51. Wu H, et al. (2007) Development of motavizumab, an ultra-potent antibody for the prevention of respiratory syncytial virus infection in the upper and lower respiratory tract. J Mol Biol 368:652-665.
52. Bedzyk W D, Johnson L S, Riordan G S, Voss E W, Jr. (1989) Comparison of variable region primary structures within an anti-fluorescein idiotype family. J Biol Chem 264:1565-1569.

We claim:

1. A method of reducing a B-cell mediated immune response to a protein comprising administering to a patient in need thereof a fusion composition comprising:
a) a first domain comprising said protein; and
b) a second domain comprising an Fc variant of a human wild-type Fc region that binds the FcγRIIb receptor with a Kd of less than about 100 nM.

2. A method according to claim 1 wherein said protein is a therapeutic protein.

3. A method according to claim 1 wherein said protein is an autoantigen.

4. A method according to claim 1 wherein said protein is an allergen.

5. A method according to claim 1 wherein said fusion is a direct protein fusion.

6. A method according to claim 1 wherein said fusion includes a protein linker.

7. A method of reducing a B-cell mediated immune response to a therapeutic antibody comprising administering to a patient in need thereof a variant therapeutic antibody, wherein the Fc domain of said therapeutic antibody comprises an Fc variant of a human wild-type Fc region that binds the FcγRIIb receptor Kd of less than about 100 nM.

8. A method of treating a B-cell mediated autoimmune disease comprising administering to a patient in need thereof a fusion composition comprising:
a) an autoantigen first domain associated with said autoimmune disease; and
b) a second domain comprising an Fc variant of a human wild-type Fc region that binds the FcγRIIb receptor Kd of less than about 100 nM.

9. In a method of treatment comprising the administration of a therapeutic antibody to a patient in need thereof, the improvement comprising administering a variant therapeutic antibody wherein the Fc region of said antibody is an Fc variant of a human wild-type Fc region that binds the FcγRIIb receptor with a Kd of less than about 100 nM.

10. In a method of treatment comprising the administration of a therapeutic protein to a patient in need thereof, the improvement comprising administering a fusion therapeutic protein comprising:
a) said therapeutic protein; and
b) an Fc variant of a human wild-type Fc region that binds the FcγRIIb receptor with a Kd of less than about 100 nM.

11. A method according to any of claim 1, 7, 8, 9 or 10 wherein said Fc variant comprises an amino acid substitution selected from the group consisting of 234D, 234E, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E, wherein numbering is according to the EU index.

12. A method according to claim 11 wherein said Fc variant comprises an amino acid substitution selected from the group consisting of 235Y, 236D, 239D, 266M, 267E, 268D, 268E, 328F, 328W, and 328Y wherein numbering is according to the EU index.

13. A method according to claim 11 wherein said Fc variant comprises amino acid substitutions selected from the group consisting of 235Y/267E, 236D/267E, 239D/268D, 239D/267E, 267E/268D, 267E/268E, and 267E/328F, wherein numbering is according to the EU index.

14. A fusion composition comprising:
a) a first fusion protein comprising a protein selected from the group consisting of an autoantigen, an allergen and a non-antibody therapeutic protein; and
b) a second fusion protein comprising an Fc variant of a human wild-type Fc region that binds the FcγRIIb receptor with a Kd of less than about 100 nM.

15. A composition according to claim 11 wherein said Fc variant comprises an amino acid substitution selected from the group consisting of 234D, 234E, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E, wherein numbering is according to the EU index.

16. A composition according to claim 15 wherein said Fc variant comprises amino acid substitutions selected from the group consisting of 235Y/267E, 236D/267E, 239D/268D, 239D/267E, 267E/268D, 267E/268E, and 267E/328F, wherein numbering is according to the EU index.

17. A nucleic acid encoding the composition of claim 14.

18. A host cell comprising the nucleic acid of claim 17.

19. A method of making the composition of claim 14 comprising culturing the host cell of claim 18 under conditions where said composition is produced.


SEQUENCE LISTING (18 sequences)

SEQ ID NO: 1 - protein, 214 aa, Homo sapiens
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 451 aa, Homo sapiens
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 451 aa, Artificial Sequence
  Fig 4 Adalimumab anti-TNF variant (S267E/L328F) heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 222 aa, Homo sapiens
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 221 aa, Homo sapiens
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 6 - protein, 222 aa, Homo sapiens
CPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 7 - protein, 222 aa, Homo sapiens
CPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 222 aa, Homo sapiens
CPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 125 aa, Artificial Sequence
  Fig 10 Extracellular domain of human MOG protein
GQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPG

SEQ ID NO: 10 - protein, 357 aa, Artificial Sequence
  8434 huMOG(ECD)-Fc(IgG1)
GQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 357 aa, Artificial Sequence
  8436 huMOG(ECD)-Fc(IIbE)
GQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGEPKSSDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 357 aa, Artificial Sequence
  8438 huMOG(ECD)-Fc(KO)
GQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGEPKSSDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 253 aa, Artificial Sequence
  8435_muMOG(35-55)-Fc(IgG1)
MEVGWYRSPFSRVVHLYRNGKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 253 aa, Artificial Sequence
  8437_muMOG(35-55)-Fc(IIbE)
MEVGWYRSPFSRVVHLYRNGKEPKSSDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 5 aa, Artificial Sequence
  gycine-serine polymer linker
GSGGS

SEQ ID NO: 16 - protein, 5 aa, Artificial Sequence
  gycine-serine polymer linker
GGGGS

SEQ ID NO: 17 - protein, 4 aa, Artificial Sequence
  gycine-serine polymer linker
GGGS

SEQ ID NO: 18 - protein, 5 aa, Artificial Sequence
  gycine-serine polymer linker
GSSSS